SEQUENCE LISTING (26 sequences)

SEQ ID NO: 1 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 2 - DNA, 536 bp, Homo sapiens
atggctgtcctgcagaaatctatgagtttttcccttatggggactttggccgccagctgcctgcttctcattgccctgtgggcccaggaggcaaatgcgctgcccatcaacacccggtgcaagcttgaggtgtccaacttccagcagccgtacatcgtcaaccgcacctttatgctggccaaggaggccagccttgcagataacaacacagacgtccggctcatcggggagaaactgttcgaggagtcagtgctaaggatcagtgctacctgatgaagcaggtgctcaacttcaccctggaagacgttctgctcccccagtcagacaggttccagccctacatgcaggaggtggtgcctttcctgaccaaactcagcaatcagctcagctcctgtcacatcagcggtgacgaccagaacatccagaagaatgtcagaaggctgaaggagacagtgaaaaagcttggagagagtggagagatcaaagcgattggggaactggacctgctgtttatgtctctgagaaatgcttgcgtc

SEQ ID NO: 3 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 4 - DNA, 546 bp, Homo sapiens
atgaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaac

SEQ ID NO: 5 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 6 - DNA, 32 bp, Artificial Sequence
  Synthetic construct
ggtggtggatccatggctgtcctgcagaaatc

SEQ ID NO: 7 - DNA, 32 bp, Artificial Sequence
  Synthetic construct
ggtggtccatgggacgcaagcatttctcagag

SEQ ID NO: 8 - DNA, 32 bp, Artificial Sequence
  Synthetic construct
ggtggtggatccatgaacaacaggtggatcct

SEQ ID NO: 9 - DNA, 33 bp, Artificial Sequence
  Synthetic construct
ggtggtccatgggttttggaagtttctggtaag

SEQ ID NO: 10 - DNA, 30 bp, Artificial Sequence
  Synthetic construct
gcgctctagaatggctgtcctgcagaaatc

SEQ ID NO: 11 - DNA, 29 bp, Artificial Sequence
  Synthetic construct
gcgctctagaatgaacaacaggtggatcc

SEQ ID NO: 12 - DNA, 33 bp, Artificial Sequence
  Synthetic construct
gcgccccgggttacttgtacagctcgtccatgc

SEQ ID NO: 13 - DNA, 80 bp, Artificial Sequence
  Synthetic construct
tcaaaccaccaggaccaagcgctgaaagacgacgctttctgcttaattcacctaatgggttggtttgatccatgaactgg

SEQ ID NO: 14 - DNA, 30 bp, Artificial Sequence
  Synthetic construct
attccttggacttcatttactgggtttaac

SEQ ID NO: 15 - DNA, 30 bp, Artificial Sequence
  Synthetic construct
ttgatatgcctcctaaatttttatctaaag

SEQ ID NO: 16 - DNA, 25 bp, Artificial Sequence
  Synthetic construct
taatatgagataatgccgactgtac

SEQ ID NO: 17 - DNA, 20 bp, Artificial Sequence
  Synthetic construct
tgaatgagtgagtcaacttg

SEQ ID NO: 18 - DNA, 80 bp, Artificial Sequence
  Synthetic construct
cgttaaaataggaaaacctttgcttaggtcaaatcgcaagctttatccgaaaacagatttagtacctgttcctgtccgat

SEQ ID NO: 19 - DNA, 26 bp, Artificial Sequence
  Synthetic construct
gctatttcttagataaagtggctgac

SEQ ID NO: 20 - DNA, 25 bp, Artificial Sequence
  Synthetic construct
tttgcttaggtcaaatcgcaagctt

SEQ ID NO: 21 - DNA, 26 bp, Artificial Sequence
  Synthetic construct
aaaattggaacatggtgtgacatgga

SEQ ID NO: 22 - DNA, 22 bp, Artificial Sequence
  Synthetic construct
ttaaactgctacgggagccttg

SEQ ID NO: 23 - DNA, 25 bp, Artificial Sequence
  Synthetic construct
taatctcgctttgattgttctatcg

SEQ ID NO: 24 - DNA, 25 bp, Artificial Sequence
  Synthetic construct
aaggaagataaatcccataagggcg

SEQ ID NO: 25 - DNA, 1271 bp, Artificial Sequence
  Synthetic construct
ggatccatggctgtcctgcagaaatctatgagtttttcccttatggggactttggccgccagctgcctgcttctcattgccctgtgggcccaggaggcaaatgcgctgcccatcaacacccggtgcaagcttgaggtgtccaacttccagcagccgtacatcgtcaaccgcacctttatgctggccaaggaggccagccttgcagataacaacacagacgtccggctcatcggggagaaactgttccgaggagtcagtgctaaggatcagtgctacctgatgaagcaggtgctcaacttcaccctggaagacgttctgctcccccagtcagacaggttccagccctacatgcaggaggtggtgcctttcctgaccaaactcagcaatcagctcagctcctgtcacatcagcggtgacgaccagaacatccagaagaatgtcagaaggctgaaggagacagtgaaaaagcttggagagagtggagagatcaaagcgattggggaactggacctgctgtttatgtctctgagaaatgcttgcgtcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgag

SEQ ID NO: 26 - DNA, 1280 bp, Artificial Sequence
  Synthetic construct
ggatccatgaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaacccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgag

(19) United States
(12) Patent Application Publication
(10) Pub. No.: US 2022/0362340 A1
(43) Pub. Date: Nov. 17, 2022

(54) COMPOSITIONS AND USES THEREOF FOR TREATING IRRADIATION-INDUCED INTESTINAL DAMAGE

(71) Applicants: UNIVERSITY OF PITTSBURGH-OF THE COMMONWEALTH SYSTEM OF HIGHER EDUCATION, Pittsburgh, PA (US); WISCONSIN ALUMNI RESEARCH FOUNDATION, Madison, WI (US)

(72) Inventors: Joel Greenberger, Sewickley, PA (US); Michael Epperly, Pittsburgh, PA (US); Xichen Zhang, Cheswick, PA (US); Jan Peter Van Pijkeren, Madison, WI (US)

(21) Appl. No.: 17/773,373

(22) PCT Filed: Oct. 30, 2020
(86) PCT No.: PCT/US2020/058105

§ 371 Date: Apr. 29, 2022

Related U.S. Application Data
(60) Provisional application No. 62/929,146, filed on Nov. 1, 2019.

(51) Int. Cl. A61K 38/20; A61K 35/747; A61K 35/741; A61K 38/21; A61K 38/19; A61K 31/4184; A61K 31/4468; A61K 31/4178; A61K 31/352; A61K 31/341; A61P 1/00

(52) U.S. Cl. CPC A61K 38/20; A61K 35/747; A61K 35/741; A61K 38/215; A61K 38/193; A61K 31/4184; A61K 31/4468; A61K 31/4178; A61K 31/352; A61K 31/341; A61P 1/00

(57) ABSTRACT

Disclosed herein are gastrointestinal tract (G1) bacteria and methods for treating or preventing an irradiation-induced intestinal damage in a subject, the methods comprising administering a G1 bacterium to the subject, wherein the G1 bacterium comprises a vector that comprises a polynucleotide encoding IL-22 and/or IFN-P, or a functional fragment thereof.

### CROSS-REFERENCE TO RELATED APPLICATIONS

This application claims the priority benefit of U.S. Provisional Application No. 62/929,146, filed Nov. 1, 2019, which is incorporated herein by reference in its entirety.

### STATEMENT REGARDING FEDERALLY SPONSORED RESEARCH OR DEVELOPMENT

This invention was made with government support under grant number AI068021 awarded by the National Institutes of Health. The government has certain rights in the invention.

### FIELD

The present disclosure relates to gastrointestinal tract (GI) bacteria and uses thereof for treating radiation-induced intestinal damage.

### BACKGROUND

Irradiation damage to tissues can occur following relatively common procedures such as irradiation applied as a cancer or other disease treatment, or uncommon events such a irradiation terrorist event or a nuclear reactor accident. The search for irradiation protectors and irradiation mitigator drugs (the latter showing effectiveness after irradiation exposure) has led to discoveries of multiple classes of therapeutic agents. [Rwigema J -CM, et al. (2011); Steinman J, et al. (2018)]. These include drugs, which block distinct irradiation-induced cell death pathways including apoptosis, necroptosis, ferroptosis, and parthanatos. [Vanden Berghe T, et al. (2014)].

Importantly, however, ionizing irradiation specifically damages the intestine and leads to the Gastrointestinal (GI) Syndrome [Asano J, et al., (2017); Wang X, et al., (2015).; Wei L, et al., (2016); Wei L, et al., (2018)]. Studies have shown that total body irradiation (TBI) at levels that exceed a normal dose that can be rescued by bone marrow transplantation (Hematopoietic Syndrome) kills intestinal crypt cells, damages the villus, and results in a systemic increase in gut bacteria, which leads to sepsis, and, ultimately, death. [Wang X, et al., (2015); Wei L, et al., (2016); Wei L, et al., (2018)]. A distinct gastrointestinal (GI) syndrome is observed after these higher TBI doses, and kills mice 5-10 days after exposure to doses in excess of 12 Gray (Gy) TBI. Animals receiving 15 Gy total abdominal irradiation die within 10 days associated with shrinkage of antimicrobial intestinal Paneth cells that, naturally, produce defensins, lysozyme, and other antimicrobial factors. [Chen J, et al., (2017); Riba A, et al., (2017); Guo X, et al., (2018); Gassier N, (2017); Dayton T L, and Clevers H, (2017); Langlands A J, et al., (2016); Bel S, et al., (2017); Rogala A R, et al., (2018); Yu S, et al., (2018)]. Loss of intestinal crypt cells, principally, lgr5+ stem cells [Zha J -M, et al., (2018)] is also detected after doses that produce death from the GI syndrome. [Wang X, et al., (2015); Wei L, et al., (2016); Wei L, et al., (2018)].

Accordingly, what are needed are compositions and methods for treating or preventing intestinal damage caused by irradiation. The compositions and methods disclosed herein address these and other needs.

### SUMMARY

Disclosed herein are gastrointestinal tract (GI) bacteria and methods for treating or preventing an irradiation-induced intestinal damage in a subject comprising administering a GI bacterium to the subject, wherein the GI bacterium comprises a vector that comprises a polynucleotide encoding IL-22 and/or IFN-β, or a functional fragment thereof. The GI bacterium can be, for example, Lactobacillus reuteri or Escherichia coli. The GI bacterium and the method disclosed herein result in surprising treatment and/or prevention of intestinal damage causes by irradiation (e.g., abdominal or total body irradiation). In some embodiments, the subject has a cancer and has received or is intended to receive an irradiation therapy.

In some aspects, disclosed herein is a method of treating an irradiation-induced intestinal damage in a subject comprising administering to the subject a therapeutically effective amount of a gastrointestinal tract (GI) bacterium, wherein the bacterium comprises a vector comprising a polynucleotide that encodes IL-22 and/or IFN-β, or a functional fragment thereof. In some embodiments, the GI bacterium is a specie of Escherichia genus or Lactobacillus genus. In some embodiments, the GI bacterium is Lactobacillus reuteri. In some embodiments, the GI bacterium is Escherichia coli. The GI bacterium can secrete IL-22 and/or IFN-β, or a functional fragment thereof. In one example, the subject has a cancer and has received or is intended to receive an irradiation therapy (e.g., total or abdominal administration). The GI bacterium can be administered to the subject via any route, and in some aspects, is an oral administration. In some embodiments, the method further comprises administering to the subject a therapeutically effective amount of an irradiation mitigator (e.g., JP4-039, Necrostatin, Baicalein, XJB-Veliparib, triphenyl-phosphonium-Veliparib, MCC950, or G-CSF).

In some aspects, disclosed herein is a gastrointestinal tract (GI) bacterium comprising a vector, wherein the vector comprises a polynucleotide that encodes IL-22 or a functional fragment thereof, and wherein the bacterium secretes IL-22 or a functional fragment thereof.

In some aspects, disclosed herein is a GI bacterium comprising a vector, wherein the vector comprises a polynucleotide that encodes IFN-β or a functional fragment thereof, and wherein the bacterium secretes IFN-β or a functional fragment thereof.

In some embodiments, the GI bacterium is a species of Escherichia genus or Lactobacillus genus. In some embodiments, the GI bacterium is Lactobacillus reuteri. In some embodiments, the GI bacterium is Escherichia coli.

### DESCRIPTION OF DRAWINGS

FIG. 1 shows the plasmid map of the pRSET-EmGFP used to transform E. Coli. The mouse IL-22 or IFN-β construct was inserted into the vector between the BamH1 and Nco1 restriction sites (arrow) forming pRSET-IL-22-EmGFP or pRSET-IFN-β-EmGFP plasmid, respectively.

FIG. 2 shows the plasmid map of pJP028_EFTu_IL22_thyA used to transform Lactobacillus reuteri for expressing mouse IL-22.

FIG. 3 shows gene expression for IFN-β and IL-22 in clones of E. coli-IFN-β and E. coli-IL-22, as demonstrated by PCR. Left 3 lanes are 3 representative clones, positive for IFN-β; right 5 lanes are 5 representative clones positive for IL-22.

FIGS. 4A-4E show secretion of IL-22 and IFN-β by E. coli cells transformed with pRSET-IL-22-GFP or pRSET-IFN-β-GFP plasmids. FIG. 4A shows a comparison of levels of IL-22 detected in culture medium collected at 48 hrs. Lined bar represents E. coli-GFP medium, white bar represents Lactobacillus-IL-22 medium (p=0.0007), black bar represents E. coli-IL-22 medium (p=0.0034). FIG. 4B shows a comparison of cell lysates from E. coli-IL-22 with Lactobacillus-rueteri-IL-22. Lined bar represents E. coli-GFP lysate, white bar represents Lactobacillus-IL-22 lysate (p=0.0089), black bar represents E. coli-IL-22 lysate (p=0.0023). FIG. 4C shows detection of IFN-β in lysates from E. coli-IFN-β (p<0.0001). FIG. 4D shows detection of IFN-β in 48 hrs medium from E. coli-IFN-β (p=0.0047). In FIGS. 4C and 4D, black bar represents E. coli group and white bar represents E. coli-IFN-β group. FIG. 4E shows biological activity of IL-22 produced by Lactobacillus-rueteri-IL-22 using the induction of IL-10 in Colo205 cells. Black bar is Colo205, lined bar is LR (p=0.0401 vs Colo205, and white bar is LR-IL-22 (p=0.0016 vs Colo205; p=0.0025 vs LR).

FIG. 5 shows GFP expression in E. coli-IL-22-GFP+cells (×1000).

FIGS. 6A-6B show histologic localization of GFP+E. coli-IL-22 to the ileum at 4 hrs after gavage and at 24 hrs after TBI of 109 E. coli-IL-22-GFP+; FIG. 6A shows cross section of ileum×20. FIG. 6B shows (inset) localization of Lactobacillus-IL-22-GFP+ fusion protein to the ileum epithelial surface at 4 hrs after gavage and at 24 hrs after TBI (×100) into 9.25 Gy irradiated C57BL/6J mice. Arrow—epithelia break (absence of actin staining), triangle—bacteria in villi near macrophages.

FIG. 7 depicts improved survival of IL-22-Lactobacillus-reuteri gavaged TBI mice (n=10). Groups of 10 mice received 9.25 Gy TBI, then 24 hrs later gavage of 100 μl of saline containing 10⁹ bacteria, or IL-22 protein delivered I.M. at 20 mg/kg in 100 or 100 μμl cyclodextrin containing 20 mg/kg JP4-039. Circle is 9.25 Gy; square is 9.25 Gy+JP4-039, p=0.0079; triangle is 9.25 Gy+IL-22 protein, p=0.0428; inverted triangle is 9.25 Gy+IL-22 lactobacillus, p=0.0114; diamond is 9.25 Gy+control lactobacillus, p=0.5201.

FIG. 8 shows superior survival after 9.25 Gy TBI of C57BL/6 mice gavaged with 100 μl saline containing 10⁹ E. coli-IL-22-GFP+ at 24 hrs or E. coli-IFN-13-GFP+ at 48 hrs (n=15). FMT (fecal microbiome transplant) of 10⁹ bacteria from 30 day survivors of 9.25 Gy by TBI. IFN-β or IL-22 protein, or JP4-039 were administered as described in the legend to FIG. 7. Circle is 9.25 Gy; filled square is JP4-039, p=0.0195; filled triangle is IL-22 (0.1 mg/kg), p=0.0428; filled diamond is IFN-β (protein), p=0.0078; inverted filled triangle is E. coli-IL-22, p=0.0004; open square is E. coli-IFN-β, p=0.0008; open diamond is FMT, p=0.6876; inverted open triangle is E. coli, p=0.8851.

FIG. 9 demonstrates significantly improved survival following gavage of IL-22-Lactobacillus-reuteri or I.M. injection of IL-22 protein (0.1 mg/kg) 24 hr after 9.25 Gy total body irradiation of C57BL/6 mice (n=10). FMT (fecal microbiome transplant had no effect on survival following TBI. Circle is 9.25 Gy, inverted filled triangle is Lactobacillus-reuteri-IL-22, p=0.004, triangle is IL-22 protein, p=0.0428, and inverted open triangle is FMT.

FIGS. 10A-10B show that gavage of Lactobacillus-reuteri-IL-22 24 hr after 15 Gy after partial body irradiation (FIG. 10A) or 19.75 Gy total abdominal irradiation (FIG. 10B) of C57BL/6 mice significantly improves survival. Control Lactobacillus reuteri had no effect on survival following 19.75 Gy total abdominal irradiation. In FIG. A the circle is 15 Gy and the square is Lactobacillus reuteri-IL-22 (p=0.0008). In FIG. B the circle is 19.75 Gy (n=10), the square is control Lactobacillus reuteri (n=10, p=0.4491) and the inverted triangle is Lactobacillus-reuteri-IL-22 (n=10, p=0.0138).

FIGS. 11A-11B demonstrate that mitigation of irradiation damage is dependent on the quantity of Lactobacillus reuteri-ILL-22 administered. C57BL/6 mice were irradiated to 9.25 Gy TBI and gavaged 24 h later with doses of Lactobacillus reuteri-IL-22 ranging from 0 to 10⁹ bacteria. No mitigation was detected unless the mice are gavaged with 10⁹ cells. Circle is no bacteria, square is 10⁶ cells, triangle is 10⁷ cells, inverted triangle is 10⁸ cells and open circle is 10⁹ cells, p=0.01.

FIG. 12 shows that Lactobacillus-reuteri-IL-22 gavage at 24 hrs after TBI (IR) rescues and preserves critical lgr5+ cells in ileum of lgr5+ GFP+ mice at day 7 (×1000), (p=00357). Mice were gavaged with LR-IL-22 as described in the legend to FIG. 7. At day 7, mice were sacrificed, ileum removed and fixed, then 20 cross-sections of ileum were scored for number of lgr5+ GFP+ intestinal stem cells. Results are the mean +/−SEM.

FIG. 13 shows that gavage of Lactobacillus reuteri-IL-22 24 hr after 9.25 Gy TBI not only protects the intestine but also has a protective effect on the bone marrow. C57BL/6 mice were irradiated to 9.25 Gy and gavaged with Lactobacillus reuteri, IL-22 protein (0.1 mg/kg) or Lactobacillus reuteri-IL-22 24 h after irradiation. The mice were sacrificed on day 3 or 5 after irradiation, bone marrow removed, and plated in methylcellulose media. Colonies of greater than 50 cells were counted 14 days later. There was a significant decrease in colonies in all irradiated mice compare to non-irradiated mice. However, mice gavaged with Lactobacillus reuteri-IL-22 had a significantly increased number of colonies compared to 9.25 Gy only on both day 3 and day 5 after irradiation, p=0.0040 and 0.0351, respectively. Open bar is 0 Gy, closed bar is 9.25 Gy, horizonal stripes are 9.25 Gy+Lactobacillus reuteri, cross stripes are 9.25 Gy +IL-22 protein, and vertical stripes are 9.25 Gy+Lactobacillus reuteri-IL-22

FIG. 14 shows significant mitigation of 9.25 Gy TBI delivered to C57BL/6 female mice by gavage of Lactobacillus-reuteri-IL-22-GFP+ (10⁹ bacteria in 100 μl saline) administered at 24, 48, or 72 hrs after irradiation (n=12). Square is Gy; inverted triangle is IL-22 Lactobacillus, 24 hr (p=0.0001); diamond is IL-22 Lactobacillus, 48 hr (p=0.0047); IL-22 Lactobacillus, 72 hr (0.0448).

FIG. 15 shows dose response of number of Lactobacillus-IL-22 (LB-IL-22), E. coli-IL-22 (EC-IL-22), or E. coli-IFN-β (EC-IFN-β) gavaged at 24 hrs after 9.25 Gy TBI relative to survival (n=10). C57BL/6 mice were irradiated to 9.25 Gy and gavaged 24 h later with Lactobacillus reuteri-IL-22, Escherichia coli-IL-22 or Escherichia coli-IFN-β at concentration of cells ranging from 10⁶ to 10⁹ cells. The best mitigation for all three bacteria was in mice gavaged with 10⁹ cells. Closed circle is 9.25 Gy, Closed square is lactobacillus reuteri-IL-22 (LR-IL-22) 10⁶ cells, closed triangle is LR-IL22 10⁷ cells, inverted closed triangle is LR-IL22 10⁸ cells, X is LR-IL22 10⁹ cells, p=0.0209, open circle is Escherichia coli-IL22 (EC-IL22) 10⁶ cells, open square is EC-IL22 10⁷ cells, p=0.0303, open triangle is EC-IL22 10⁸ cells, open circle with dot is EC-IL22 10⁹ cells, p=0.0139, open diamond is Escherichia coli-IFN-β (EC-IFNB) 10⁶ cells, small circle is EC-IFNB 10⁷ cells, star is EC-IFNB 10⁸ cells and + is EC-IFNB 10⁹ cells, p=0.0459.

FIGS. 16A and 16B show the common fecal microbiome after LD80/30 TBI in all survivors. FIG. 16A shows that each taxon in individual animals at 14 days after TBI (or day before death) in all 48 animals (n=12 per group) was correlated to, which survived to day 30 or died between days 14 and 30. The relative abundance of all taxa was transformed to prevent false correlation. Additive log-ratio (ALR) transformation was used to make each taxonomic abundance a log ratio against the background taxa. “−” represents the greatest negative and “+” positive associations (p-value≤0.10). In survivors of 9.25 Gy alone (left column=difference from pre-irradiation), there was decreased Bacteroides and Parabacteroides, and increased (+) Bacteroidales S24 7. The mitigator treated survivors showed the same results: only additional differences from rad-control are shown. G-CSF treated survivors also had decreased Akkermansia. Survivors in the G-CSF plus JP4-039 treatment group also had decreased Oscillibacter. FIG. 16B shows a survival analysis as time varying covariates, revealed a statistically significant (p-value=0.00244) association between retained Lactobacillus at day 14 (d) and survival to day 30. All individual 30 day survivors after TBI showed stabilized levels at day 14 of 9 taxa, but only Lactobacillus showed a significant correlation with survival to day 30. Pre-irradiation (Panel PT). Day 1 post-irradiation levels of Lactobacillus after 9.25 Gy levels dropped in all groups (Panel D1). No recovery of Lactobacillus at day 14 in individual mice that died before day 30 (Panel D14 NS). In contrast, individuals destined to live to day 30 all showed increased abundance of Lactobacillus (Panel D14 S) persisting to day 30 (Panel D30 S). Within each panel, the bars from left to right represent irradiated control, G-CSF treatment, combined treatment of G-CSF+JP4-039, JP4-039 treatment, and pooled data from all groups. n=12 per group.

FIGS. 17A and 17B show that intestinal level of IL-22 is reduced by TBI and restored by JP4-039 or BMT. IL-22 is produced in the GI tract by gamma delta (γσ) T cells and/or other cells stimulated by (γσ) T cells. Levels drop after 9.25 Gy TBI (ctrl, IR), but are restored by treatment with JP4-039 at 24 h (FIG. 17A) or by bone marrow transplant (BMT) at 24 hrs after TBI (FIG. 17B). In FIG. 17A, solid line is control, dashed line is JP4-039. In FIG. 17B, solid line is IR, dashed line is IR/BMT.

FIGS. 18A-18B show TBI dose-dependent reduction in functional lgr5+ stem cells and MMP-7+ Paneth cells. The ileum of lgr5+ GFP+ mice was analyzed at indicated times (0-96 h) after total body irradiation (TBI, 8 Gy (light gray bar), 12 Gy (black bar), or 15 Gy (dark gray bar)). FIG. 18A shows number of lgr5+ stem cells in cross sections. Values are mean+SEM, N=3 mice. Eight full cross sections per mouse. ***p<0.001, **p<0.01 (Student's T Test, two-tailed). FIG. 18B shows representative images of Igr5+ GFP+ (arrow) or matrix metalloproteinase-7 (MMP-7) staining of Paneth cells in their crypts. Arrow heads indicate ring structures with collapsed crypts (MMP-7 Paneth cells dysfunction). Scale Bar: 50 μM.

FIG. 19 shows that JP4-039+Lactobacillus-IL-22 gavage at 24 h is a better mitigator combination than either alone. Circle is 9.25 Gy; square is 9.25 Gy+JP4-039, p=0.0467; triangle is 9.25 Gy+IL-22 lactobacillus, p=0.0114; inverted triangle is 9.25 Gy +JP4-039+IL-22 Lactobacillus, p=0.0004.

FIG. 20 shows that genetically engineered probiotics increase survival following 19.75 Gy abdominal irradiation. Filled circle is 9.25 Gy (n=10); square is control Lactobacillus (n=5, p=0.4491); inverted triangle is IL-22 Lactobacillus (n=10, p=0.0138); diamond is E. coli-IL-22 (n=10, p=0.0473); open circle is E. coli-IFN-β (n=10, p=0.0299).

### DETAILED DESCRIPTION

Disclosed herein are compositions and methods for treating an irradiation-induced intestinal damage in a subject by administering to the subject a therapeutically effective amount of a gastrointestinal tract (GI) bacterium, wherein the bacterium comprises a vector comprising a polynucleotide that encodes IL-22 or IFN-β, or a functional fragment thereof. It is a surprising finding of the present invention that bacteria harboring the aforesaid vectors can treat irradiation-induced intestinal damage, and therefore, can be effectively used after exposure to irradiation (e.g., in cancer treatment or a radiation terrorist event) or before exposure to irradiation.

In some embodiments, the GI bacterium is Lactobacillus subtilus, Lactobacillus reuteri, or Escherichia coli. In some embodiments, the GI bacterium secrets IL-22 or IFN-β, or a functional fragment thereof. In some embodiments, the GI bacterium is administered to the subject via an oral administration. The methods described herein can also further comprise administering to the subject a therapeutically effective amount of an irradiation mitigator, wherein the irradiation mitigator is JP4-039, Necrostatin, Baicalein, XJB-Veliparib, or G-CSF. The oral administration of such GI bacterium comprising a vector comprising a polynucleotide that encodes IL-22 or IFN-β surprisingly improves the survival of irradiated animals better than administering irradiation mitigators alone.

Terms used throughout this application are to be construed with ordinary and typical meaning to those of ordinary skill in the art. However, Applicants desire that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, and the like. Administration includes self-administration and the administration by another. In some embodiments, the administration of a GI bacterium to a subject is done via oral administration.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a radiation-induced intestinal damage). The terms also encompass pharmaceutically acceptable, pharmacologically active derivatives of beneficial agents specifically mentioned herein, including, but not limited to, a bacterium, a vector, polynucleotide, cells, salts, esters, amides, proagents, active metabolites, isomers, fragments, analogs, and the like. When the terms “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, composition disclosed herein comprises a gastrointestinal tract (GI) bacterium, wherein the bacterium comprises a vector comprising a polynucleotide that encodes IL-22 or IFN-β, or a functional fragment thereof.

“Effective amount” encompasses, without limitation, an amount that can ameliorate, reverse, mitigate, prevent, or diagnose a symptom or sign of a medical condition or disorder (e.g., a radiation-induced intestinal damage). Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. The term “effective amount of a GI bacterium” or “effective amount of an irradiation mitigator” refers to an amount of a GI bacterium or an irradiation mitigator sufficient to cause some mitigation of a radiation-induced intestinal damage, and/or related symptoms or restoration of intestinal function.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gastrointestinal tract” (also referred to as “GI tract” or “digestive tract”), is a series of hollow organs joined in a long, twisting tube from the mouth to the anus. The hollow organs that make up the GI tract are the mouth, esophagus, stomach, small intestine, large intestine, and anus. The small intestine has three parts—duodenum, jejunum, and ileum. The large intestine includes the appendix, cecum, colon, and rectum. However, the term “gastrointestinal tract bacterium” or “GI bacterium” is used herein to refer to a bacterium that resides in the stomach, small intestine or large intestine, such as colon, following administration (e.g., oral administration). In some embodiments the GI bacterium localizes in the stomach, small intestine or colon. In some embodiments, the GI bacterium is selected from an Escherichia genus or a Lactobacillus genus. In some embodiments, the GI bacterium is Lactobacillus subtilus. In some embodiments, the GI bacterium is Lactobacillus reuteri. In some embodiments, the GI bacterium is Escherichia coli.

An “intestinal damage”, as used herein, refers to a disruption of the homeostasis of any tissue (epithelial, connective, nervous or muscle) of any organ or compartment of the gastrointestinal tract of a mammal The intestinal damage can be a consequence of an endogenous disruption of the homeostasis of any tissue of the gastrointestinal tract, such as a cancer. Alternatively, or additionally, the intestinal damage can be a consequence of an exogenous disruption of the homeostasis of any tissue of the gastrointestinal tract. “Irradiation-induced intestinal damage” used herein refers to a disruption of the homeostasis of any tissue (epithelial, connective, nervous or muscle) of any organ or compartment of the gastrointestinal tract due to irradiation, for example, due to irradiation therapy, nuclear reactor accident or irradiation terrorist event.

As used herein, the term “intestinal stem cell” and “ISC” refers to a multipotent stem cell, such as a Lgr5+ cell.

As used herein, the term “progenitor cell” in reference to an intestinal cell refers to multipotent cells that may give rise to differentiated cells of the small or large intestine, such as a columnar cell and a goblet cell.

As used herein, the term “crypt cell” in reference to a cell refers to a multipotent stem cell found in the crypt area of the intestine.

As used herein, the term “Paneth cell” or “Davidoff's Cell” refers to a specialized type of epithelial cell found in the small intestine and the appendix. A Paneth's cell or Paneth cell is derived from an intestinal stem cell.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

“Graft-versus-host disease” or “GVHD” refers to an acute and chronic condition resulting from allogeneic hematopoietic cell transplantation (or bone marrow transplantation) in which tissues of the host, most frequently the skin, liver and intestine, are damaged by lymphocytes from the donor. The risk and severity of this immune-mediated condition are directly related to the degree of mismatch between a host and the donor of hematopoietic cells. “Bone marrow transplant” refers to a procedure that infuses healthy blood-forming stem cells into a subject's body to replace the damaged or diseased bone marrow. A bone marrow transplant is also called a stem cell transplant. A bone marrow transplant is usually done after chemotherapy and/or radiation is complete.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Therapeutically effective amount” refers to the amount of a compound such as a GI bacterium comprising a vector encoding IL-22 or INF-β that will elicit the biological or medical response of a tissue, system, animal, or human that is being sought by the researcher, veterinarian, medical doctor or other clinician over a generalized period of time. In some embodiments, a desired response is improvement of an intestinal damage caused by irradiation. In other embodiments, a desired response is reduction of an intestinal damage caused by irradiation. In some embodiments, a desired response is the preservation of Igr5+ cells in small intestine, a decrease of intestinal inflammation, an increase in anti-inflammatory cytokine IL-10 production, and/or an increased survival of a subject. In some instances, a desired biological or medical response is achieved following administration of multiple dosages of the composition to the subject over a period of days, weeks, or years. The therapeutically effective amount will vary depending on the compound such as the GI bacterium comprising a vector encoding IL-22 or INF-β, the disorder or conditions and its severity, the route of administration, time of administration, rate of excretion, drug combination, judgment of the treating physician, dosage form, and the age, weight, general health, sex and/or diet of the subject to be treated.

The therapeutically effective amount of the GI bacterium comprising a vector encoding IL-22 or INF-β compositions described herein can be determined by one of ordinary skill in the art and includes exemplary dosage amounts for a mammal of from about 0.5 to about 200 mg/kg of body weight of active composition per day, which can be administered in a single dose or in the form of individual divided doses, such as from 1 to 4 times per day. Alternatively, the dosage amount can be from about 0.5 to about 150 mg/kg of body weight of active composition per day, about 0.5 to 100 mg/kg of body weight of active compound per day, about 0.5 to about 75 mg/kg of body weight of active compound per day, about 0.5 to about 50 mg/kg of body weight of active composition per day, about 0.5 to about 25 mg/kg of body weight of active composition per day, about 1 to about 20 mg/kg of body weight of active composition per day, about 1 to about 10 mg/kg of body weight of active composition per day, about 20 mg/kg of body weight of active composition per day, about 10 mg/kg of body weight of active composition per day, or about 5 mg/kg of body weight of active composition per day.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of an intestinal damage following a subject receiving irradiation, or even before a subject receives irradiation), during early onset (e.g., upon initial signs and symptoms of an intestinal damage), after an established development of an intestinal damage, or at the stage of severe intestinal damage. Prophylactic administration can occur for several minutes to months prior to the manifestation of an irradiation-induced intestinal damage.

In some instances, the terms “treat,” “treating,” “treatment,” and grammatical variations thereof, include mitigating an irradiation-induced intestinal damage, and/or related symptoms or restoration of intestinal function in a subject as compared with prior to treatment of the subject or as compared with incidence of such symptom in a general or study population.

“Vector” used herein means, in respect to a nucleic acid sequence, a nucleic acid sequence comprising a regulatory nucleic acid sequence that controls the replication of an expressible gene. A vector may be either a self-replicating, extrachromosomal vector or a vector which integrates into a host genome. Alternatively, a vector may also be a vehicle comprising the aforementioned nucleic acid sequence. A vector may be a plasmid, bacteriophage (isolated, attenuated, recombinant, etc.). A vector may comprise a double-stranded or single-stranded DNA, RNA, or hybrid DNA/RNA sequence comprising double-stranded and/or single-stranded nucleotides. In some embodiments, the vector is a plasmid.

### Compositions

Disclosed herein are compositions for use in the treatment of an irradiation-induced intestinal damage in a subject comprising administering to the subject a therapeutically effective amount of a gastrointestinal tract (GI) bacterium, wherein the bacterium comprises a vector comprising a polynucleotide that encodes IL-22 or IFN-β, or a functional fragment thereof.

“IL-22” refers herein to a polypeptide that, in humans, is encoded by the IL22 gene. IL-22 is a cytokine that is involved in mediating cellular inflammatory responses. In some embodiments, the IL-22 polypeptide is that identified in one or more publicly available databases as follows: HGNC: 14900, Entrez Gene: 50616, Ensembl: ENSG00000127318, OMIM: 605330, UniProtKB: Q9GZX6. In some embodiments, the IL-22 polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. The IL-22 polypeptide of SEQ ID NO:1 may represent an immature or pre-processed form of mature IL-22, and accordingly, included herein are mature or processed portions of the IL-22 polypeptide in SEQ ID NO: 1. In some embodiments, the IL-22 polynucleotide comprises the sequence of SEQ ID NO: 2, or a polynucleotide sequence having at or greater than about 80%, about 85%, about 90%, about 95%, or about 98% homology with SEQ ID NO: 2, or a polynucleotide comprising a portion of SEQ ID NO: 2. In other embodiments, the IL-22 polynucleotide or polypeptide is that described in U.S. Pat. No. 10,376,563, which is incorporated by reference in its entirety, or a polynucleotide or polynucleotide sequence having at or greater than about 80%, about 85%, about 90%, about 95%, or about 98% homology with and IL-22 polynucleotide or polypeptide disclosed therein.

“IFN-β” refers herein to a polypeptide that, in humans, is encoded by the IFNB1 gene. IFN-β is a type 1 interferon that has anti-viral, antibacterial and anticancer activities. IFN-β binds to the type 1 IFN receptor. In some embodiments, the IFN-β polypeptide is that identified in one or more publicly available databases as follows: HGNC: 5434, Entrez Gene: 3456, Ensembl: ENSG0000017185,5 OMIM: 147640, UniProtKB: P01574. In some embodiments, the IFN-β polypeptide comprises the sequence of SEQ ID NO: 3, or a polypeptide sequence having at or greater than about 80%, about 85%, about 90%, about 95%, or about 98% homology with SEQ ID NO: 3, or a polypeptide comprising a portion of SEQ ID NO: 3. The IFN-β polypeptide of SEQ ID NO: 3 may represent an immature or pre-processed form of mature IFN-β, and accordingly, included herein are mature or processed portions of the IFN-β polypeptide in SEQ ID NO: 3. In some embodiments, the IFN-β polynucleotide comprises the sequence of SEQ ID NO: 4, or a polynucleotide sequence having at or greater than about 80%, about 85%, about 90%, about 95%, or about 98% homology with SEQ ID NO: 4, or a polynucleotide comprising a portion of SEQ ID NO: 4.

As noted above, the vectors described herein can be a nucleic acid sequence comprising a regulatory nucleic acid sequence that controls the replication of an expressible gene. In some embodiments, a vector comprising a promoter operably linked to a second nucleic acid (e.g., polynucleotide encoding IL-22 and/or IFN-β, or a functional fragment thereof) may include a promoter that is heterologous to the second nucleic acid (e.g., IL-22 and/or IFN-β, or a functional fragment thereof) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). It should be understood herein that the vector of any aspects described herein can further comprise a promoter, an enhancer, an antibiotic resistance gene, a thyA gene, and/or an origin, which can be operably linked to one or more of the above noted polynucleotides encoding IL-22 and/or IFN-β, or a functional fragment thereof. In some embodiments, the IL-22 vector comprises a sequence at least 80% (for examples, at least 85%, 90%, 95%, 98%, or 99%) identical to SEQ ID NO: 25. In some embodiments, the IFN-β vector comprises a sequence at least 80% (for examples, at least 85%, 90%, 95%, 98%, or 99%) identical to SEQ ID NO: 26.

It should be understood and contemplated herein that plasmid may have the thyA gene encoding thymidylate synthase deleted, and cloned thyA on the plasmid backbone encoding IL-22. Accordingly, in some embodiments, the vector comprises a thyA gene. In some embodiments, the thyA gene is on the plasmid backbone encoding IL-22 or IFN-β. In some embodiments, the vector does not comprise a thyA gene. In some embodiments, the vector does not comprise an antibiotic-resistant gene. In some embodiments, the vector comprises an antibiotic-resistant gene. The vectors, plasmids, and the methods of making thereof are also described more fully in U.S. Pat. 10,376,563; van Pijkeren J -P et al., 2012; Alexander L M et al., 2019; Fu X et al., 2000; and Hendrikx T et al., 2018, which are incorporated herein by reference for all purposes.

It should also be understood and contemplated herein that the GI bacterium can secrete and/or release a polypeptide, such as IL-22 or IFN-β, or a functional fragment thereof. The bacterium and/or vector may be engineered to produce and release the polypeptide. As used herein, “release” used with respect to the bacterium releasing the polypeptide refers to disposing the polypeptide outside the bacterium upon lysis of the bacterium. “Secretion” used herein refers to a process by which substances are produced and discharged from a live bacterial cell. Elements for engineering a bacterium to secrete a polypeptide are well known in the art. Typical elements include a signal peptide-encoding a sequence placed upstream of—and in-frame with—the coding sequence of the polypeptide to be secreted. The sequences of a large number of signal peptides for bacteria are known in the art. Exemplary signal peptide sequences are available at www.cbs.dtu.dk/services/SignalP/. Elements for inducing a bacterium to lyse include lytic proteins, which can be expressed from a bacterium through recombinant engineering. Some exemplary elements can be found in U.S. Pat. No. 10,376,563; van Pijkeren J -P et al., 2012; Alexander L M et al., 2019; Fu X et al., 2000; and Hendrikx T et al., 2018, all of which are incorporated herein by reference for all purposes.

Accordingly, included herein are GI bacterium comprising the herein described vectors that encode IL-22 and/or IFN-β, or a functional fragment thereof. As noted above, the term “gastrointestinal tract bacterium” or “GI bacterium” refers to a bacterium that resides in the stomach, small intestine or large intestine. The present invention therefore includes a GI bacterium comprising a herein described vector that resides or localizes in the small intestine and/or the large intestine following administration (e.g., oral administration). In some embodiments, the GI bacterium resides or localizes in the duodenum, jejunum, or ileum of the small intestine. Accordingly, included herein are GI bacteria that reside or localize in the duodenum. Included herein are GI bacteria that reside or localize in the jejunum. Included herein are GI bacteria that reside or localize in the ileum. In some embodiments, the GI bacterium resides or localizes in the colon. Accordingly, included herein are GI bacteria that reside or localize in the ascending colon. Included herein are GI bacteria that reside or localize in the transverse colon. Included herein are GI bacteria that reside or localize in the descending colon. Included herein are GI bacteria that reside or localize in the sigmoid colon.

The release and/or secretion of IL-22 and/or IFN-β that occurs close in proximity to intestinal epithelial cells can confer longer therapeutic effect to such intestinal epithelial cells. Therefore, disclosed herein are compositions and methods of treating an irradiation-induced intestinal damage in a subject comprising administering to the subject a therapeutically effective amount of a gastrointestinal tract (GI) bacterium, wherein the bacterium comprises a vector comprising a polynucleotide that encodes IL-22 or IFN-β, or a functional fragment thereof, and wherein the GI bacterium resides in an area of a colon or a small intestine following administration.

Exemplary GI bacteria include species of the lactic acid bacteria group, which are an order of gram-positive, low-GC, acid-tolerant, generally nonsporulating, nonrespiring, either rod-shaped (bacilli) or spherical (cocci) bacteria that share common metabolic and physiological characteristics. Representative homolactic lactic acid bacteria genera comprises Lactococcus, Enterococcus, Streptococcus, Pediococcus, and group I lactobacilli. Lactobacillus is known to localize in the jejunum and ilium of the small intestine. Therefore, in some embodiments, the GI bacterium is a species of a Lactobacillus genus. In some embodiments, the GI bacterium may include species of lactic acid bacteria other than species of a Lactococcus genus. In some embodiments, the GI bacterium is a species of a Lactococcus genus.

Exemplary species from the Lactobacillus genus include L. acetototerans, L. acidifarinae, L. acidipiscis, L. acidophilus, L. agilis, L. algidus, L. atimentarius, L. amytolyticus, L. amylophilus, L. amylotrophicus, L. amylovorus, L. animatis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. delbrueckii, L. delbrueckii subsp. butgaricus, L. delbrueckii subsp. lactis, L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. hammesii, L. hamsteri, L. harbinensis, L. hayakitensis, L. helveticus, L. hitgardii, L. homohiochii, L. iners, L. ingluviei, L. intestinalis, L. jensenii, L. johnsonii, L. katixensis, L. kefiranofaciens, L. kefiri, L. kimchii, L. kitasatonis, L. kunkeei, L. leichmannii, L. lindneri, L. malefermentans, L. mati, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracollinoides, L. parafarraginis, L. parakefiri, L. paratimentarius, L. paraplantarum, L. pentosus, L. perolens, L. plantarum, L. pontis, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. subtilus, L. thailandensis, L. ultunensis, L. vaccinostercus, L. vaginalis, L. versmoldensis, L. vini, L. vitulinus, L. zeae, and L. zymae. In some embodiments, the GI bacterium used in the method of treating radiation-induced intestinal damage of any aspects disclosed herein is L. subtilus. In some embodiments, the GI bacterium used in the method of treating irradiation-induced intestinal damage of any aspects disclosed herein is L. reuteri.

In some embodiments, exemplary GI bacteria include species of the Escherichia genus, which is a genus of gram-negative, non-spore-forming, facultatively anaerobic, rod-shaped bacteria from the family Enterobacteriaceae. Escherichia genus comprises E. albertii, E. coli, E. fergusonii, E. hermannii, E. marmotae, and E. vulneris. E. coli is shown to localize in the colon. Therefore, in some embodiments, the GI bacterium is E. coli. Accordingly, the GI bacterium used in the method of treating irradiation-induced intestinal damage of any aspects disclosed herein can be E. coli. In some embodiments, the GI bacterium used in the method of treating irradiation-induced intestinal damage of any aspects disclosed herein can be a species of Escherichia genus other than E. coli, such as E. albertii, E. fergusonii, E. hermannii, E. marmotae, or E. vulneris.

The data disclosed herein shows an alteration of GI bacteria after total body irradiation. Abundance of certain bacteria genera (e.g., Lactobacillus, Roseburia, and Akkermansia) in fecal pellets at day 14 after irradiation is correlated with survival at day 30 of an experimental animal that receives small molecule radiation mitigators. The current disclosure indicates that species of such bacterial genera can be used to treat an irradiation-induced intestinal damage.

Akkermansia is a genus in the phylum Verrucomicrobia. It can reside in human intestine. The Akkermansia genus comprises A. glycaniphila and A. muciniphila. Accordingly, in some embodiments, the GI bacterium is a species of Akkermansia genus, such as A. glycaniphila or A. muciniphila. In some embodiments, the GI bacterium used in the method of treating irradiation-induced intestinal damage of any aspects disclosed herein is A. muciniphila. In some embodiments, the GI bacterium used in the method of treating irradiation-induced intestinal damage of any aspects disclosed herein is A. glycaniphila.

Roseburia refers to a genus of butyrate-producing, Gram-positive anaerobic bacteria reside in the human colon and are members of the phylum Firmicutes. Roseburia genus comprises R. cecicola, R. faecis, R. hominis, R. intestinalis, and R. inulinivorans. Accordingly, the GI bacterium can be a species of the Roseburia genus, such as R. cecicola, R. faecis, R. hominis, R. intestinalis, or R. inulinivorans.

Accordingly, disclosed herein are compositions for treating an irradiation-induced intestinal damage in a subject comprising a therapeutically effective amount of a GI bacterium, wherein the bacterium comprises a vector comprising a polynucleotide that encodes IL-22 or IFN-β, or a functional fragment thereof. In some embodiments, the GI bacterium resides in an area of a colon or a small intestine of a subject following administration of the GI bacterium composition to the subject, wherein the GI bacterium can be of a species of any of above-stated genus, including, for example, Escherichia genus, Lactobacillus genus, Roseburian genus, and/or Akkermansia genus, and wherein the GI bacterium can release and/or secrete IL-22 and/or IFN-β, or functional fragment thereof.

The compositions of the present invention may take any form, and in some embodiments, take the form of tablet, pill or capsule for oral administration.

### Methods of Treatment

The current disclosure demonstrates the surprising finding that administering a GI bacterium (e.g., Lactobacillus subtilus, Lactobacillus reuteri, or E. coli) that releases and/or secretes IL-22 or IFN-β to a subject having irradiation damage results in a treatment of the irradiation damage. Further, the irradiation mitigating effect of the bacterium producing the IL-22 or IFN-β is greater than that achieved with administration of IL-22 or IFN-β alone. Therefore, provided herein are methods of treating an irradiation-induced intestinal damage in a subject comprising administering to the subject a therapeutically effective amount of a gastrointestinal tract (GI) bacterium, wherein the bacterium comprises a vector comprising a polynucleotide that encodes IL-22 or IFN-β, or a functional fragment thereof. The GI bacterium, vectors, and IL-22 and IFN-β compositions used in these methods can be any of those described above or below. In some embodiments, the vector comprises a polynucleotide that encodes IL-22 or a functional fragment thereof. In some embodiments, the vector comprises a polynucleotide that encodes IFN-β or a functional fragment thereof. In some embodiments, the vector comprises a polynucleotide that encodes both IL-22 and IFN-β, or a functional fragment thereof.

As noted above, the GI bacterium used herein preferably resides or localizes in the small intestine and/or large intestine, such as colon, following administration (e.g., oral administration). In such administrations, the release and/or secretion of IL-22 and/or IFN-β can confer longer therapeutic effect to the intestinal epithelial cells due to release or secretion of the IL-22 and/or IFN-β in close proximity to intestinal epithelial cells. Therefore, disclosed herein are methods of treating an irradiation-induced intestinal damage in a subject comprising administering to the subject a therapeutically effective amount of a gastrointestinal tract (GI) bacterium, wherein the bacterium comprises a vector comprising a polynucleotide that encodes IL-22 and/or IFN-β, or a functional fragment thereof, and wherein the GI bacterium resides in an area of a colon or a small intestine following administration. In some embodiments, the GI bacterium is Escherichia coli. In some embodiments, the GI bacterium can be the species of a genus selected from the group consisting of Escherichia genus, Lactobacillus genus, Roseburian genus, and/or Akkermansia genus. In some embodiments, the GI bacterium is a species of Escherichia genus or Lactobacillus genus. In some embodiments, the GI bacterium is Lactobacillus subtilus. In some embodiments, the GI bacterium is Lactobacillus reuteri. In some embodiments, the GI bacterium is E. coli.

The bacterium or the composition described herein can be administered to the subject via any route including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques. In some embodiments, routes of administration of the bacterium or the composition includes oral administration, rectal administration, and parenteral administration (intravenous, intramuscular, or subcutaneous). In some embodiments, the route of administration of the bacterium disclosed herein is oral administration.

In some embodiments, oral administration of a GI bacterium (e.g., Lactobacillus subtilus, Lactobacillus reuteri, or E. coli) releasing and/or secreting IL-22 or IFN-β, or a functional fragment thereof increases the levels of IL-22 or IFN-β, or a functional fragment thereof in a colon or a small intestine, preserves Igr5+ cells in small intestine, decreases intestinal inflammation, increases anti-inflammatory cytokine IL-10 production, and/or improves survival of a subject.

In some embodiments, the GI bacterium disclosed herein can stimulate an immune response in the abdomen of a subject having a cancer. The GI bacterium can be used as a immunostimulant and an intestinal radioprotector/mitigator. In some embodiments, the GI bacterium comprises a vector encoding IL-22. In some embodiments, the GI bacterium comprises a vector encoding IFN-β.

It should be understood that irradiation, a common therapy for cancers, such as malignancies in the abdomen and pelvis, can cause severe damage to the lining of the gastrointestinal (GI) tract, which can be composed of rapidly dividing intestinal epithelial cells. Accordingly, disclosed herein is a method of treating irradiation-induced intestinal damage in a subject, wherein the subject has a cancer and has received or is intended to receive irradiation therapy. In some embodiments, the irradiation therapy comprises total body irradiation or abdominal irradiation. In some embodiments, the irradiation therapy is total body irradiation. In some embodiments, the irradiation therapy is abdominal irradiation. In some embodiments, the cancer is selected from the group consisting of colon cancer, kidney cancer, liver cancer, gastric cancer, pancreatic cancer, cervical cancer, ovarian cancer, and adnexal/pelvic mass.

In some embodiments, disclosed herein is a method of treating irradiation-induced intestinal damage in a subject, wherein the subject has graft-versus-host-disease (GVHD). As noted above, “Graft-versus-host disease” or “GVHD” refers to an acute and chronic condition resulting from allogeneic hematopoietic cell transplantation (or bone marrow transplantation) in which tissues of the host, most frequently the skin, liver and intestine, are damaged by lymphocytes from the donor. Bone marrow transplant refers to a procedure that infuses healthy blood-forming stem cells into a subject's body to replace the damaged or diseased bone marrow. Bone marrow transplants can benefit people with a variety of both cancerous (malignant) and noncancerous diseases (such as certain autoimmune diseases and blood disorders), including, for example, Acute leukemia, Adrenoleukodystrophy, Aplastic anemia, Bone marrow failure syndromes, Chronic leukemia, Hemoglobinopathies, Hodgkin's lymphoma, Immune deficiencies, Inborn errors of metabolism, Multiple myeloma, Myelodysplastic syndromes, Neuroblastoma, Non-Hodgkin's lymphoma, Plasma cell disorders, POEMS syndrome, Primary amyloidosis, and sickle cell anemia. Chemotherapy and/or radiation is usually applied for removing the diseased bone marrow and white blood cells from the subject before bone marrow transplant. Intestinal damage can be resulted from radiation, chemotherapy or allogeneic response. Accordingly, in some embodiments, disclosed herein is a method of treating radiation-induced intestinal damage in a subject, wherein the subject has graft-versus-host-disease (GVHD) after irradiation therapy. In some embodiments, the irradiation therapy comprises total body irradiation or abdominal irradiation. In some embodiments, the irradiation therapy is total body irradiation. In some embodiments, the irradiation therapy is abdominal irradiation. In some embodiments, the subject has a disease selected from the group consisting of Acute leukemia, Adrenoleukodystrophy, Aplastic anemia, Bone marrow failure syndromes, Chronic leukemia, Hemoglobinopathies, Hodgkin's lymphoma, Immune deficiencies, Inborn errors of metabolism, Multiple myeloma, Myelodysplastic syndromes, Neuroblastoma, Non-Hodgkin's lymphoma, Plasma cell disorders, POEMS syndrome, Primary amyloidosis, and sickle cell anemia.

As noted above, “irradiation-induced intestinal damage” refers herein to a disruption of the homeostasis of any tissue (epithelial, connective, nervous or muscle) of any organ or compartment of the gastrointestinal tract due to irradiation. Ionizing irradiation damages the intestinal barrier, kills intestinal crypt cells, damages villi, and results in a systemic increase in gut bacteria, which can lead to sepsis, and, ultimately, death. Shrinkage of antimicrobial intestinal Paneth cells that, naturally, produce defensins, lysozyme and other antimicrobial factors, loss of intestinal crypt cells, principally Lgr5+ intestinal stem cells, is also detected after irradiation doses that produce death from gastrointestinal syndrome. Accordingly, each of the damages to the intestinal barrier, crypt cells, villi, Paneth cells, and Lgr5+ intestinal stem cells, increased inflammation of GI tract, systemic increase of gut bacteria, and sepsis are included within the definition of “irradiation-induced intestinal damage.”

The disclosed methods can be performed any time prior to the onset of an irradiation-induced intestinal damage. In one aspect, the disclosed methods can be employed 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours; 60, 48, 36, 30, 24, 18, 15, 12, 10, 9, 8, 7, 6, 5, 4, 3, or 2 minutes prior to the irradiation therapy; or 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours; 60, 48, 36, 30, 24, 18, 15, 12, 10, 9, 8, 7, 6, 5, 4, 3, or 2 minutes after irradiation therapy, prior to the onset of a radiation-induced intestinal damage; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of a radiation-induced intestinal damage.

Dosing frequency for the vector or the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

The GI bacterium disclosed herein may be administered in any amount effective to introduce the polypeptide in the bloodstream of the subject. Exemplary amounts include from about 1×10³ to about 1×10⁵, from about 1×10⁵ to about 1×10¹³, from about 1×10⁷ to about 1×10¹¹, from about 1×10⁶ to about 1×10⁹ colony forming units (CFU). In some embodiments, the dosage is about 1×10⁹, about 1×10⁸, about 1×10⁷, or about 1×10⁶ CFUs.

Pharmaceutically acceptable excipients or carriers are well known to those of skill in the art and may include cellulose and its derivatives (e.g. sodium carboxymethylcellulose, sodium ethylcellulose, cellulose acetate, etc.), gelatin, speckstone, solid lubricating agent (e.g. stearic acid, magnesium stearate), calcium sulphate, plant oil (e.g. pea oil, sesame oil, peanut oil, olive oil, etc.), polyols (e.g. propylene glycol, glycerol, mannitol, sorbitol, etc.), emulsifier (e.g. Tween®), wetting agent (e.g. sodium lauryl sulfate), colorant, flavoring agent, stabilizer, anti-oxidant, antiseptic, pyrogen-free water, etc.

In some embodiments, the method of treating an irradiation-induced intestinal damage of any preceding aspects further comprises administering to the subject a therapeutically effective amount of one or more irradiation mitigators, wherein the combination of the bacterium comprising a IL-22 and/or IFN-β vector and the irradiation mitigator produces a synergistic effect. In some embodiments, the irradiation mitigator is JP4-039, Necrostatin, Baicalein, XJB-Veliparib, triphenyl-phosphonium-Veliparib, MCC950, or G-CSF. Exemplary chemical structures of JP4-039, Necrostatin, Baicelaein and MCC950 are provided below.

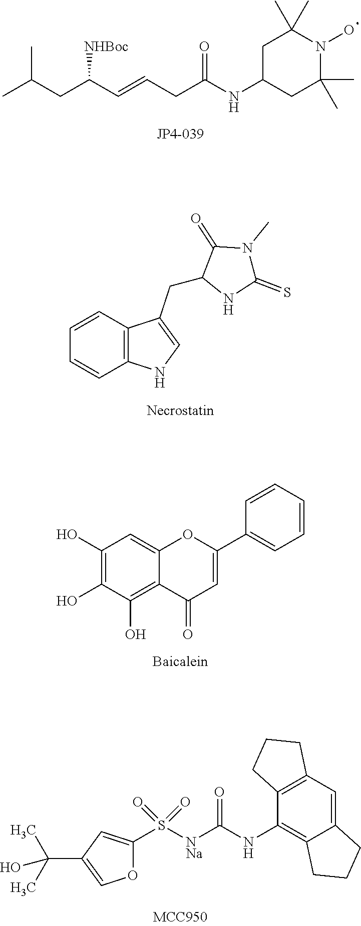

The chemical structure of XJB-Veliparib is described in Krainz T, et al., Synthesis and evaluation of a mitochondria-targeting Poly(ADP-ribose) Polymerase-1 inhibitor. ACS, Chem Biol, 13(10): 2868-2878, 2018, which is incorporated herein by reference for all purposes.

The chemical structure of triphenyl-phosphonium-Veliparib is described in Stoyanovsky DA, et al., Targeting and activation of NO donors in mitochondria. Peroxidase metabolism of (2-Hydroxyamino-Vinyl)-Triphenyl-Phosphonium by cytochrome c releases NO and protects cells from apoptosis. FEBJ Letters 583: 2000-2005, 2009, which is incorporated herein by reference for all purposes.

“Granulocyte colony-stimulating factor”, G-CSF, or GCSF refers herein to a polypeptide that, in humans, is encoded by the GSF3 gene. In some embodiments, the G-CSF polypeptide is that identified in one or more publicly available databases as follows: HGNC: 2438, Entrez Gene: 1440, Ensembl: ENSG00000108342, OMIM: 138970, UniProtKB: P09919. In some embodiments, the G-CSF polypeptide comprises the sequence of SEQ ID NO: 5, or a polypeptide sequence having at or greater than about 80%, about 85%, about 90%, about 95%, or about 98% homology with SEQ ID NO: 5, or a polypeptide comprising a portion of SEQ ID NO: 5. The G-CSF polypeptide of SEQ ID NO: 5 may represent an immature or pre-processed form of mature G-CSF, and accordingly, included herein are mature or processed portions of the G-CSF polypeptide in SEQ ID NO: 5.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

### Radiation Affects the GI Tract Cells and Cytokines and Treatment with Small Molecule Radiation Mitigators Alters the GI Microbiome

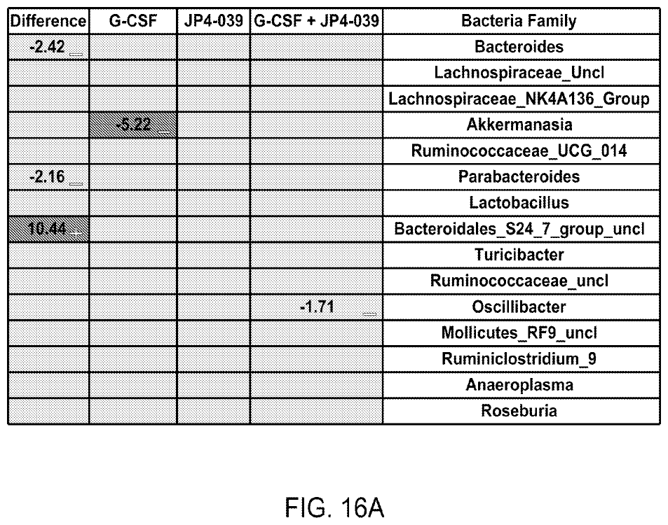

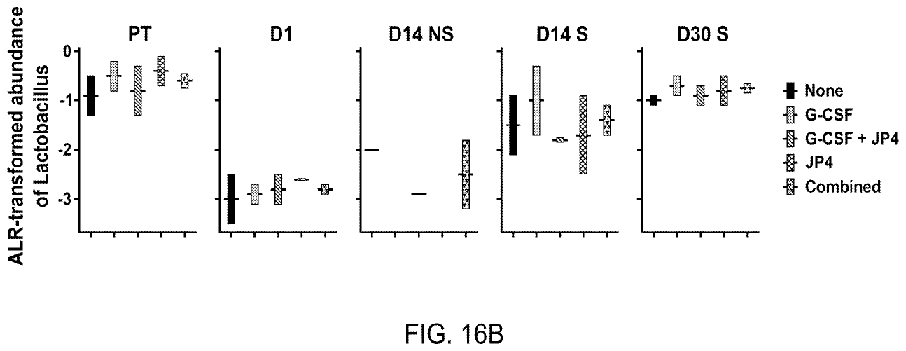

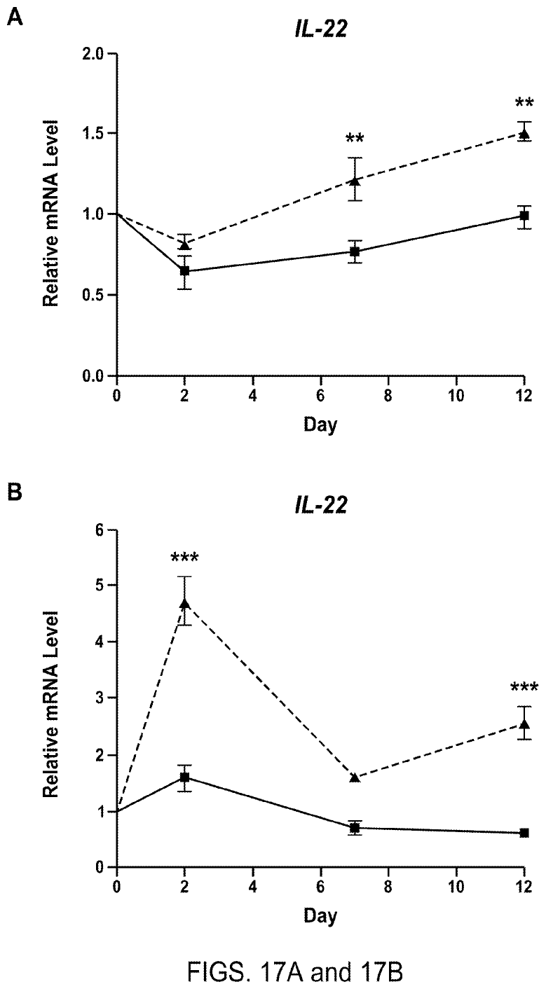

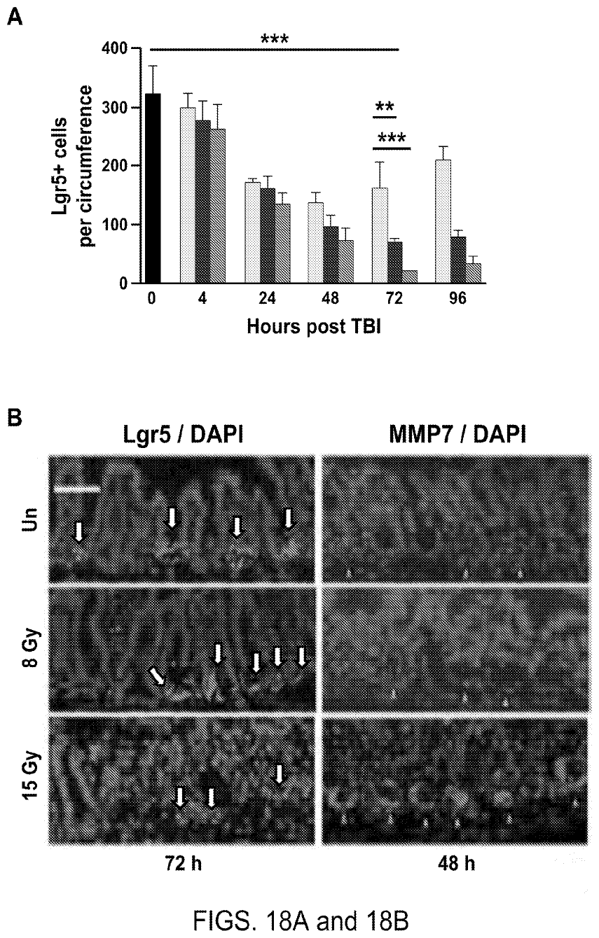

The data disclosed herein reveal that mitigation of TBI by JP4- 039, G-CSF, or both agents altered the intestinal microbiome and all survivors including irradiated controls stabilized their intestinal Lactobacillus (FIG. 16). Specifically, stabilization of intestinal Lactobacillus after TBI was uniformly detected in fecal samples at 14 days after TBI from animals destined to survive to 30 days in both control irradiated and mitigator treated mice (FIG. 16). It was also determined that levels of the anti-inflammatory cytokine IL-22 dropped after irradiation, and either JP4-039 (FIG. 17A) or bone marrow transplant (BMT) restored IL-22 levels (FIG. 17B). Data further show that irradiation damaged lgr5+ stem cells and Paneth cells (FIG. 18). Numbers of MMPI Paneth cells and lgr5+ intestinal stem cells in lgr+ GFP+ mice decreased at 72 h after 8 Gy TBI (FIG. 18).

### Example 2

### Methods and Materials

Mice and Animal Care. Female C57BL/6NTac (Taconic Biosciences, Wayne, Pa.) and C57BL/6 lgr5+ green fluorescent protein tagged (5-6) lgr5+ mice were housed 5 per cage and fed standard laboratory chow and deionized drinking water according to University of Pittsburgh IACUC regulations.

Irradiation. Total body irradiation was delivered with a cesium-137 Gamma Cell irradiator with filters removed at a dose rate of 310 cGy/min, beam flatness, homogeneity, and analysis of spectrum of gamma ray precise dosimetry were carried out. Mice were irradiated in a plexiglass pie plate that contains 12 sections. Five mice were placed in each pie plate. The mice were not placed in adjacent sections, but had one or two empty sections between mice so that the mice would not shield the other mice in the pie plate. Thermoluminescent (TLD) readings according to published methods. [Steinman J, et al., Improved total- body irradiation survival by delivery of two radiation mitigators that target distinct cell death pathways. Radiat Res 189(1): 68-83, 2018.]

Bacterial Strains and Growth Conditions. E. coli DH5α (Thermo Fisher) was used for subcloning and plasmid amplification. E. coli BL21 (New England Biolabs) was used as the expression host. E. coli was grown in Luria-Bertani (LB) broth at 37° C. with 200 rpm shaking. Ampicillin (100 μg/mL) was added if needed for selection. Lactobacillus reuteri strains were cultured at 37° C. under static conditions in de Man Rogosa Sharpe (MRS) medium. Successful and stable transformations were assayed for both intracellular and secreted (into the culture broth) of IL-22. By PCR we confirmed cloning of the gfp gene, while GFP protein production was confirmed by both observation of green color in bacterial transformants. In E. coli, selection of bacterial transformants was carried out using the metabolic marker construct ampicillin. Successful transformants were assayed for both intracellular and secreted (into the culture broth) of IL-22. GFP was determined by both observation of green color in bacterial transformants and also by PCR assay for the transgene for green fluorescent protein.

Plasmid Construction, Expression, and Plasmid Transfection Reagents. Plasmid pRSET-EmGFP purchased from Thermo Fisher (Cat. #V35320) was used as the E. coli BL21 expression vector. The gene coding Interleukin-22 (IL-22) (GeneBank: NM_016971.2) and interferon-beta (IFN-β, GeneBank: K00020.1) were amplified from a cDNA library of C57BL/6 mouse bone marrow stromal cells. Both of IL-22 and IFN-β were fused to the GFP protein gene on vector pRSET-EmGFP between BamH1 and Nco1 restriction sites. IL-22 gene was amplified by the primers: IL22-Forward (SEQ ID NO: 6): 5′-GGT GGT GGA TCC ATG GCT GTC CTG CAG AAA TC; IL22-Reverse (SEQ ID NO: 7): 5′-GGT GGT CCA TGG GAC GCA AGC ATT TCT CAG AG. IFN-β was amplified by the primers: IFN-Forward (SEQ ID NO: 8): 5′-GGT GGT GGA TCC ATG AAC AAC AGG TGG ATC CT; IFN-Reverse (SEQ ID NO: 9): 5′-GGT GGT CCA TGG GTT TTG GAA GTT TCT GGT AAG. Both of IL22 and IFN-β with GFP gene were amplified by PCR respectively and cloned into pHT43 vector Xba1/Xma1 site. The primers were used as: IL22-GFP-Forward (SEQ ID NO: 10): 5′-GCGC TCT AGA ATG GCT GTC CTG CAG AAA TC; IFNb-GFP-Forward (SEQ ID NO: 11): 5′-GCGC TCT AGA ATG AAC AAC AGG TGG ATC C; Both GFP-Reverse (SEQ ID NO: 12): 5′-GCGC CCC GGG TTA CTT GTA CAG CTC GTC CAT GC.

The plasmids were first transformed into E. coli DH5a and the expression cassettes were verified by DNA sequencing. The plasmids pRSETEmGFP/IL22 or pRSETEmGFP/IFN-beta were transformed into E. coli BL21 by heating transformation generating E. coli BL21 strains expressing IL-22 or IFN-β.

Construction of LRΔthyA::rpoB(H488R)/pIL-22-thyA. By single-stranded DNA recombineering, the thyA was inactivated in a rifampicin-resistant derivative of Lactobacillus reuteri VPL1014 (LR:rpoB(H488R)) to yield LRΔthyA (Rif®), as described previously [van Pijkeren J -P et al., 2012; Alexander LM et al., 2019] (Tables 1 and 2). From here onwards, this strain is referred to as LR*. The gene providing chloramphenicol resistance in the vector pVPL31126 was replaced with the thyA gene derived from L. reuteri VPL1014 via blunt-end ligation (T4 DNA ligase: Fisher Scientific) and transformed into LR* by electroporation to construct LR*/pIL-22-thyA, as described previously [Alexander L M et al., 2019; Fu X et al., 2000; Hendrikx T et al., 2018]. LR* harboring the previously constructed vector pCtl-thyA served as an empty vector control [Alexander L M et al., 2019]. Escherichia coli EC1000 was used as an intermediate cloning host.

### Strain Table

TABLE 1
Bacterial strains and plasmids used in this study

| | Characteristics† | Source/Reference* |
|---|---|---|
| Strains | | |
| (Name/VPL) | | |
| E. coli | Derivative of E. coli MC1000 in which repA is | Leenhouts K, 1996 |
| EC1000 | integrated in chromosome | |
| L. reuteri | Derivative of L. reuteri ATCCPTA6475 | Biogaia |
| VPL1014 | | |
| LR* or | Rifampicin resistant mutant with inactivated | Alexander L M, 2019 |
| VPL31132 | thyA gene generated by oVPL236 and | |
| | oVPL1670, respectively | |
| Plasmids | | |
| pVPL2042 | EmR, pNZ8048 derivative. Cm marker was | Van Pijkeren Lab stock |
| | replaced by Em marker | |
| pVPL31126 | pJP028 derivative, pIL-22 expression vector | Hendrikx T, 2018 |
| pVPL31168 | pJP028 derivative, pIL-22-ThyA | This work |
| pVPL31134 | pJP028 derivative, pCtl:ThyA | Alexander L M, 2019 |

TABLE 2
Oligonucleotides used in this study

| oligo | | |
|---|---|---|
| name | sequence (5′-3′)† | Target/comment# |
| oVPL236 | (SEQ ID NO: 13) | Recombineering oligo |
| | tcaaaccaccaggaccaagcgctgaaagacgacgctttctgcttaat | for L. reuteri RpoB* |
| | tcacctaatgggttggtttgatccatgaactgg | mutant |
| oVPL329 | (SEQ ID NO: 14) attccttggacttcatttactgggtttaac | Rev, for pJP028 |
| | | insertion screening |
| OVPL362 | (SEQ ID NO: 15) ttgatatgcctcctaaatttttatctaaag | Rev, for pJP028 |
| | | insertion screening |
| oVPL363 | (SEQ ID NO: 16) taatatgagataatgccgactgtac | Fwd, for pJP028 |
| | | insertion screening |
| oVPL736 | (SEQ ID NO: 17) tgaatgagtgagtcaacttg | Fwd, amplified pMutL |
| | | of L. reuteri |
| oVPL1670 | (SEQ ID NO: 18) | Recombineering oligo |
| | cgttaaaataggaaaacctttgcttaggtcaaatcgcaagctttat | for ΔthyA |
| | ccgaaaacagatttagtacctgttcctgtccgat | |
| oVPL1671 | (SEQ ID NO: 19) gctatttcttagataaagtggctgac | Fwd, for screening of |
| | | ΔthyA |
| oVPL1672 | (SEQ ID NO: 20) tttgcttaggtcaaatcgcaagctt | Rev, for screening of |
| | | ΔthyA |
| oVPL1673 | (SEQ ID NO: 21) aaaattggaacatggtgtgacatgga | Rev, for screening of |
| | | ΔthyA |
| oVP11725 | (SEQ ID NO: 22) ttaaactgctacgggagccttg | Rev, amplified pMut- |
| | | thyA |
| oVPL2351 | (SEQ ID NO: 23) taatctcgctttgattgttctatcg | Rev, amplified pJP028 |
| | | backbone omitting |
| | | CmR- cassette |
| oVPL2352 | (SEQ ID NO: 24) aaggaagataaatcccataagggcg | Fwd, amplified |
| | | pJP028 backbone |
| | | omitting CmR- |
| | | cassette |

ELISA Assays. IL-22 ELISA kit (Thermo Fisher: BMS6022, Waltham, Mass.) and IFN-β ELISA kit (Thermo Fisher: 42400-1, Waltham, Mass.) were used to quantify the expression level of IL-22 or IFN-β by following the company's instruction. Lysates of transformed bacterial strains (lysed by sonication) and culture medium were used to determine IL-22 levels.

IL-22 Biological Activity Assay. The activity of both E. coli IL-22 and Lactobacillus-IL-22 was determined by measuring secretion of interleukin-10 in the Colo 20 (ATCC CCL-222) human colon carcinoma cell line. IL-10 production is positively regulated by IL-22. Colo 205 cells were maintained in RPMI-1640 supplemented with 10% FBS, 1% pen-strep and 1% L-glutamine. Cells were seeded in 6-well plates at a density of 1×10⁶ cells/well several hours before adding IL-22. At 24 h after addition of IL-22, the amount of IL-10 in the cell-free medium from the Colo 205 cell was determined by IL-10 ELISA kit (Thermo Fisher: BMS215-2, Waltham, Mass.). The positive control IL-22 (using levels of 1 pg/ml to 1 μg/ml were tested) and was obtained from Peprotech (Rocky Hill, N.J.). Selection of bacterial transformation was carried out using the metabolic marker construct ampicillin. Successful and stable transformations were assayed for both intracellular and secreted (into the culture broth) of each cytokine. Synthesis of GFP fusion product was determined by both observation of green color in bacterial transformants and also by PCR assay for the transgene for green fluorescent protein.

Dose Response Curve of Number of Gavaged Lactobacillus-reuteri, or E. coli Producing IL-22 or IFN-β. Mice were irradiated to 9.25 Gy TBI, which took 13-14 minutes, and were then 24 h later gavaged with 100 μL saline solution containing Lactobacillus reuteri-IL-22, or E. coli-IL-22, or at 48 hrs post irradiation E. coli-IFN-β (based on the known time of requirement for IFN-β by lgr5+ cells at this time). All groups of mice received 100 μL saline, which contained varying numbers of bacteria ranging from 10⁹, 10⁸, 10⁷, to 10⁶ bacteria (n=10 animals per group).

Assay for Transgene IL-22 and IFN-β, and GFP Fusion Protein Transgenes by Polymerase Chain Reaction (RT-PCR). Bacterial strains were evaluated by recombinant DNA reagents in assays for transgene insertion using standard procedures for PCR. Primers used to detect transgene IL-22, IFN-β, and gfp were obtained from (Integrated DNA Technologies, Austin, Tex., USA) and have been described above.

Assays for Intestinal Paneth, Goblet, and lgr5+ Stem Cells. These assays were carried out using antibodies and assay conditions according to published methods [Wei L, et al., Inhibition of CDK4/6 protects against radiation-induced intestinal injury in mice. J Clin Invest 126(11): 4076-4086, 2016; Wei L, et al., The GS-nitroxide JP4-039 improves intestinal barrier and stem cell recovery in irradiated mice. Scientific Rep 8: 2072, 2018].

Administration of Small Molecule Radiation Mitigator, IL-22 Protein, L. reuteri Lacking IL-22, or Gavaged Fecal Microbiome. As a control for probiotic mitigators, the GS-nitroxide, JP4-039 [Rwigema J -CM, et al., Two strategies for the development of mitochondrial-targeted small molecule radiation damage mitigators. Int J Radiat Oncol Biol Phys 80(3): 860-868, 2011], was prepared and administered according to published methods, I.M. at 20 mg/kg in 50 μL of 30% cyclodextrin aqueous [Epperly M W, et al., Evaluations of different formulations and routes for the delivery of the ionizing radiation mitigation GS-nitroxide (JP4-039). In Vivo 32: 1009-1023, 2018]. Cyclodextrin alone has no radiomitigative effect. IL-22 protein was I.P.

administered at 0.1 mg/kg. 10⁹ L. reuteri lacking IL-22 was administered in 100 μl/saline. A similar number of bacteria from feces obtained from 30-day survivors of 9.25 Gy TBI was gavaged. Fecal microbiome transplant (FMT) was carried out by delivery by gavage in 100 μL saline; 10⁹ bacteria from the feces of 30-day survivors of 9.25 Gy TBI of C57BL/6 female mice.

Immunostaining and Imaging. Imaging of intestinal barrier function was carried out using multicolor staining techniques according to published methods [Tyurina Y Y, et al., Redox (phospho)lipidomics of signaling in inflammation and programmed cell death. J Leukocyte Biol 106: 57-81, 2019]. In particular, imaging of GFP+ bacteria location relative to breeches in intestinal keratin barrier were carried out according to published methods [Tyurina Y Y, et al., Redox (phospho)lipidomics of signaling in inflammation and programmed cell death. J Leukocyte Biol 106: 57-81, 2019].

### Example 3

### Creation of IL-22 and IFN-β Producing Lactobacillus and E. Coli

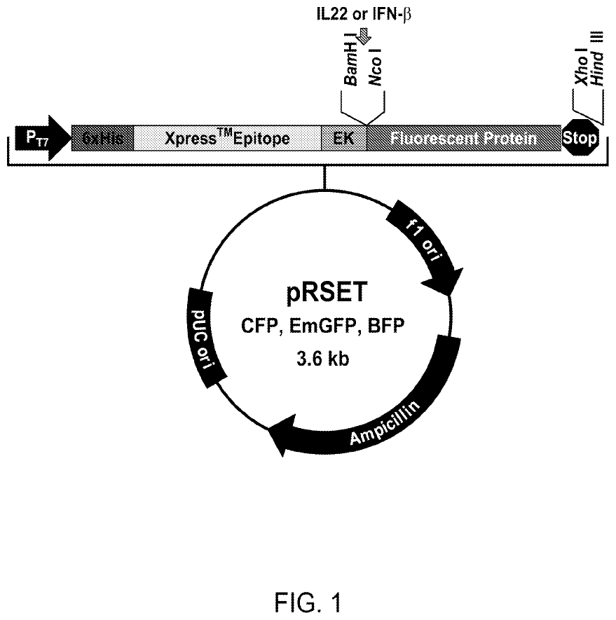

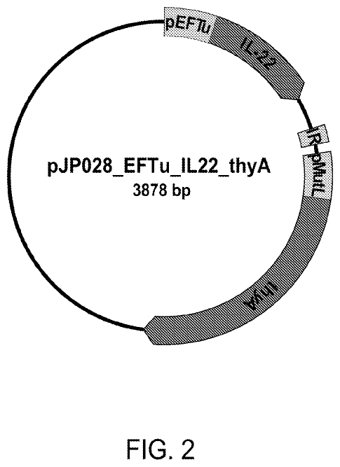

The plasmid constructs used to transform E. coli (EC) are shown in FIG. 1. Insertion sites for the transgene for IL-22 or IFN-β are shown, restriction enzyme locations for insertion of transgenes are shown. The construction of the Lactobacillus-IL-22 bacterial strain has been previously described [Hendrikx T, et al., Bacteria engineered to produce IL-22 in intestine induce expression of REG3G to reduce ethanol-induced liver disease in mice. Gut Epub ahead of print Nov. 17, 2018].

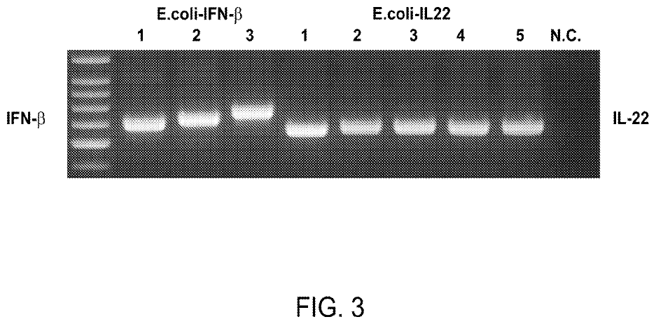

Gene sequences for the GFP fusion proteins for IL-22 and IFN-β are shown in SEQ ID NO:25 AND SEQ ID NO:26. Transformed bacterial sublines and positive clones were selected for ampicillin (100 μg/ml) and chloramphenicol (10 μg/ml) resistance. Confirmation of insert location in bacterial substrains was carried out by Polymerase Chain Reaction (FIG. 3). FIG. 3 demonstrates E. coli subclones containing the IFN-β or IL-22 transgenes.

### Example 3

### Confirmation of Production of IL-22 or IFN-β Protein

Representative clones of each of the two strains of genetically modified E. coli, one of, which secretes IL-22 and the other secreting IFN-β were chosen for biological evaluation. As a positive control for these new bacterial strains, Lactobacillus-reuteri producing IL-22 was expanded for comparison [Hendrikx T, et al., Bacteria engineered to produce IL-22 in intestine induce expression of REG3G to reduce ethanol-induced liver disease in mice. Gut Epub ahead of print Nov. 17, 2018]. As controls for biological assays, E. coli, which were transformed with the pRSET-GFP plasmid were studied.

The current study used a derivative of a previously constructed Lactobacillus reuteri strain that produces IL-22. To ensure plasmid stability without the need for antibiotic selection, the essential gene thyA, which encodes thymidylate synthase, was deleted and cloned on the plasmid backbone encoding IL-22. [Hendrikx T, et al., Bacteria engineered to produce IL-22 in intestine induce expression of REG3G to reduce ethanol-induced liver disease in mice. Gut Epub ahead of print Nov. 17, 2018.] The plasmid will never be lost in the absence of antibiotic selection, because it provides ThyA in trans. [Alexander L M, et al., Exploiting prophage-mediated lysis for biotherapeutic release by Lactobacillus reuteri. Appl Environ Microbiol 85: e02335-18, 2019. PMID: 30683744. DOI: 10.1128/AEM.02335-18].

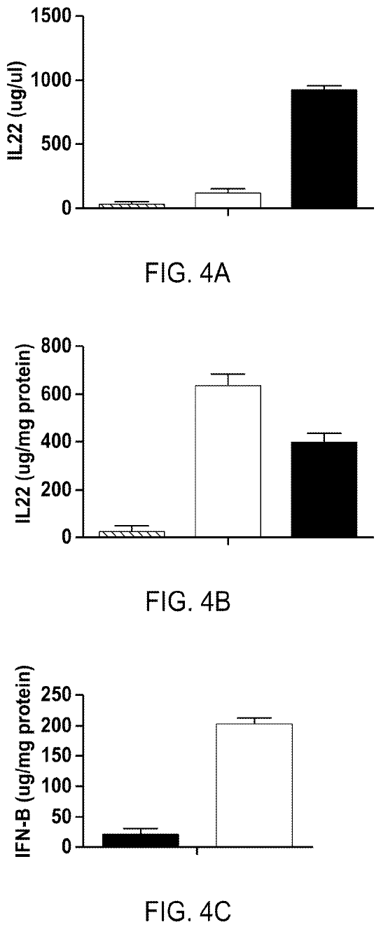

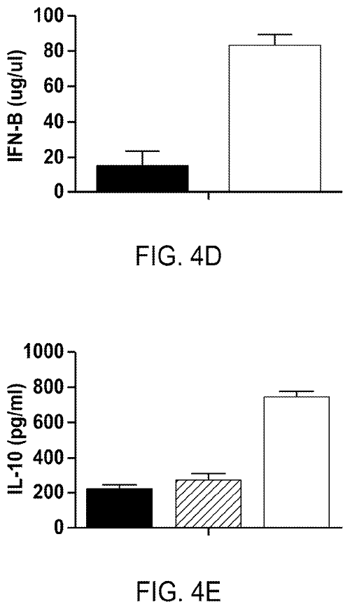

Bacteria were grown in standard culture broth and both cytocentrifuged bacteria cell pellets, and culture medium were assayed for IL-22 and IFN-β by ELISA assay. As shown in FIG. 4A, the lysates from E. coli-IL-22 and Lactobacillus-IL-22 bacteria contained IL-22, while the E. coli did not. The medium from which the three bacteria were grown was assayed by IL-22 ELISA. The medium in which the E. coli-IL-22 was positive for IL-22, but the Lactobacillus-IL-22 and E. coli-GFP did not (FIG. 4B). These data indicate that the E. coli-IL-22 secreted the cytokine, while Lactobacillus-reuteri-IL-22 did not.

IFN-β ELISA assay was performed on the cell lysates (FIG. 4C) or medium (FIG. 4D) in which the E. coli-IFN-β or E. coli-IFN-β, but not the E. coli-GFP indicating that the E. coli-IFN-β can secrete the IFN-β protein.

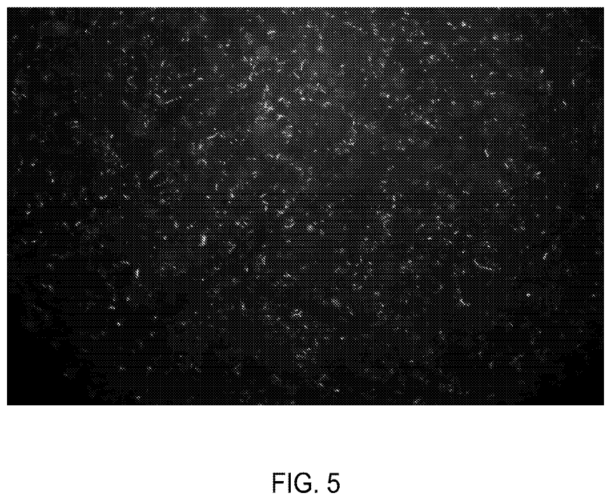

As shown in FIG. 4E, cell culture medium containing secreted IL-22 from E. coli-IL-22, but not Lactobacillus-reuteri (intracellular IL-22) induced IL-10 production in target colon cancer cell line in vitro. This shows that the IL-22 secreted from the E. coli-IL-22 was biologically active. Observation of the E. coli-IFN-β (FIG. 5A) and E. coli-IL-22 (FIG. 5B) by fluorescent microscopy demonstrated that the cells were GFP positive.

### Example 4

### Localization of Il-22-GFP and IFN-β-GFP to the Intestine

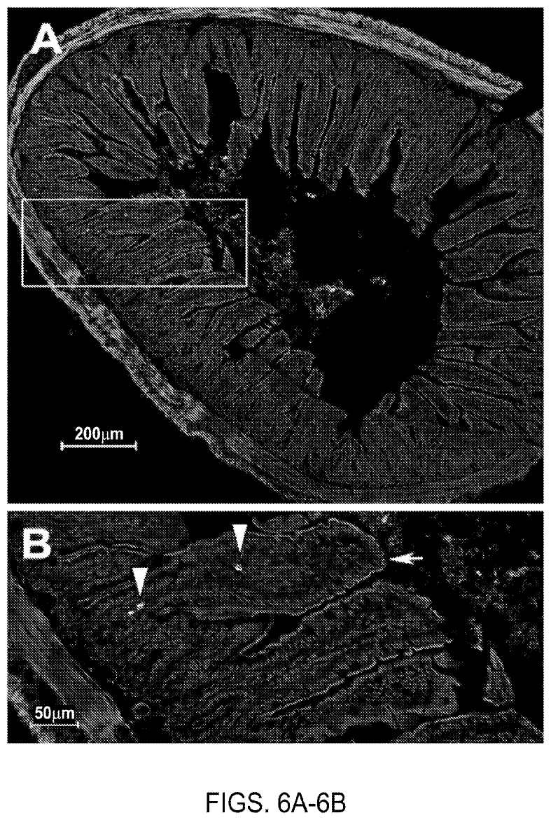

Localization of GFP+ fusion protein cytokine to the intestine was carried out by imaging sections of jejunum, ileum, and colon. As shown in FIG. 6, E. coli-IL-22-GFP+ was observed in jejunum and ileum 24 hrs after gavage and colon. Bacteria were cleared from the colon by day 5 after gavage. Similar localization was seen with L. reuteri-IL-22-GFP+ (data not shown).

### Example 5

### Radiation Mitigation by Lactobacillus-IL-22

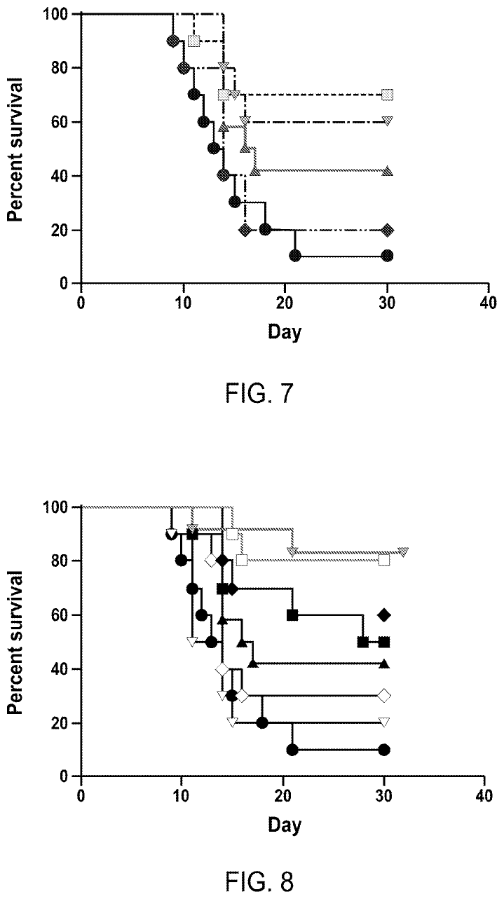

C57BL/6NTac mice were irradiated to 9.25 Gy total body irradiation and gavaged with Lactobacillus reuteri-IL-22 or Lactobacillus reuteri harboring the empty vector control. Additional controls included animals subjected to IP injection of recombinant IL-22 protein or the radiation mitigator, JP4-039. Compared to the animals that only received TBI, we observed increased survival at 30 days after TBI in mice administered Lactobacillus-IL-22 (60%), IL-22 protein (40%), or JP4-039 (70%) (FIG. 7). There was no significant increase in survival following gavage of Lactobacillus reuteri wild type compared to control 9.25 Gy TBI (10%). Thus, probiotic-mediated delivery of IL-22 increased the survival of animals exposed to TBI at levels that are comparable to the radiation-mitigation compound JP4-039.

### Example 5

### Radiation Mitigation by E. Coli-IL-22 and E. Coli-IFN-β

Irradiated C57BL/6NTac mice were administered by gavage E. coli-IL-22 or E. coli-GFP at 24 hr post-irradiation or E. coli-IFN-β at 48 hr after irradiation and followed for development of hematopoietic syndrome. Mice administered with either E. coli-IL-22 or E. coli-IFN-β had increased survival after TBI (FIG. 8). E. coli-GFP or Lactobacillus control gavage resulted in no increase in survival.

### Example 6

### Radiation Mitigation by L. Reuteri-IL22 is Superior to IL-22 Protein or Fecal Microbiome Transplant (FMT)

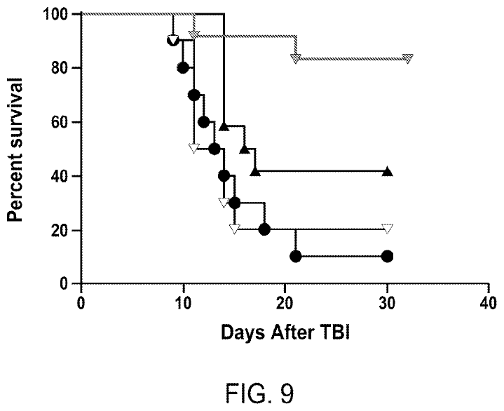

C57B1/6NTac mice were irradiated 9.25 Gy total body irradiation and then gavaged with L. reuteri-IL22, injected I.M. with IL-22 protein (0.1 mg/kg) or had a FMT (FIG. 9). Mice having a FMT showed no increase is survival compared to irradiation only mice. Mice treated with IL-22 protein or L. reuteri-IL22 had a significantly increased survival with the mice gavaged with L. reuteri-IL22 being superior to IL22 protein.

### Example 7

### L. reuteri-IL22 Mitigates Partial Body Irradiation

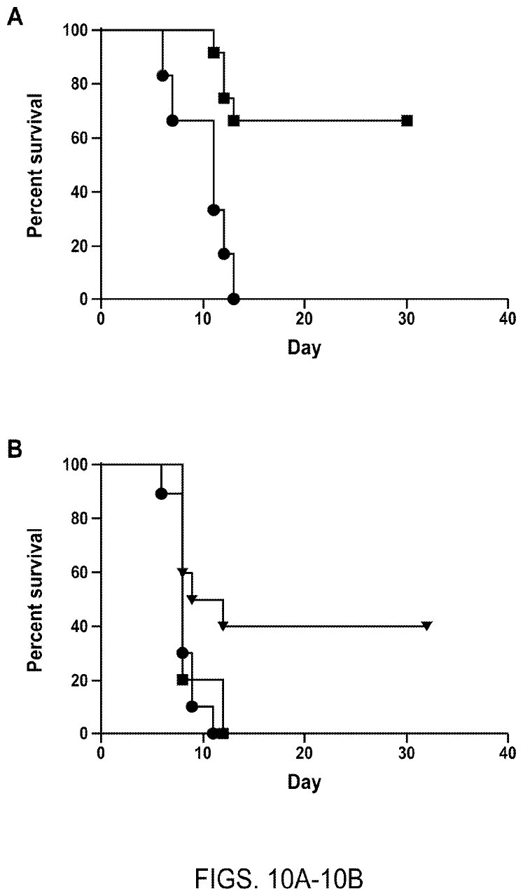

C57BL/6NTac mice were irradiated to 15 Gy partial body irradiation where one leg is shielded from irradiation while the rest of the body is irradiated. This method preserves the bone marrow and hematopoietic system so that you determine whether damage to the intestine. As seen in FIG. 10, L. reuteri-IL22 was able to mitigate partial body irradiation to the intestine.

### Example 8

### Mitigation of Total Abdominal Irradiation by L. Reuteri-IL22

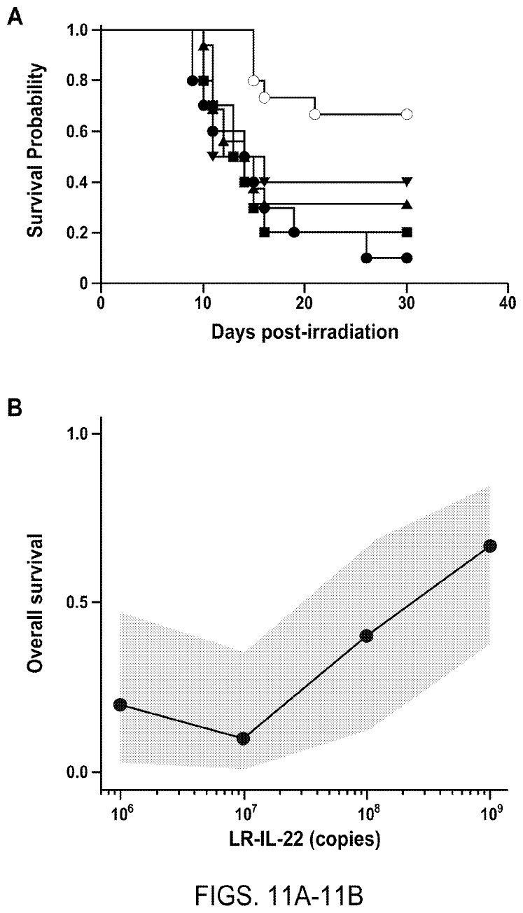

C57BL/6NTac mice were irradiated toe 19.75 Gy total abdominal irradiation and gavaged 24 h later with L. reuteri or L. Reuteri-IL22. Mice gavaged with L. reuteri was not able to mitigate total abdominal irradiation while mice gavaged with L. reuteri-IL22 had a significant increase is survival (FIG. 11). This demonstrates that L. reuteri-IL22 mitigates intestinal damage resulting from total abdominal irradiation.

### Example 9

### Histopathologic Analysis of Subsets of Intestinal Crypt Cells

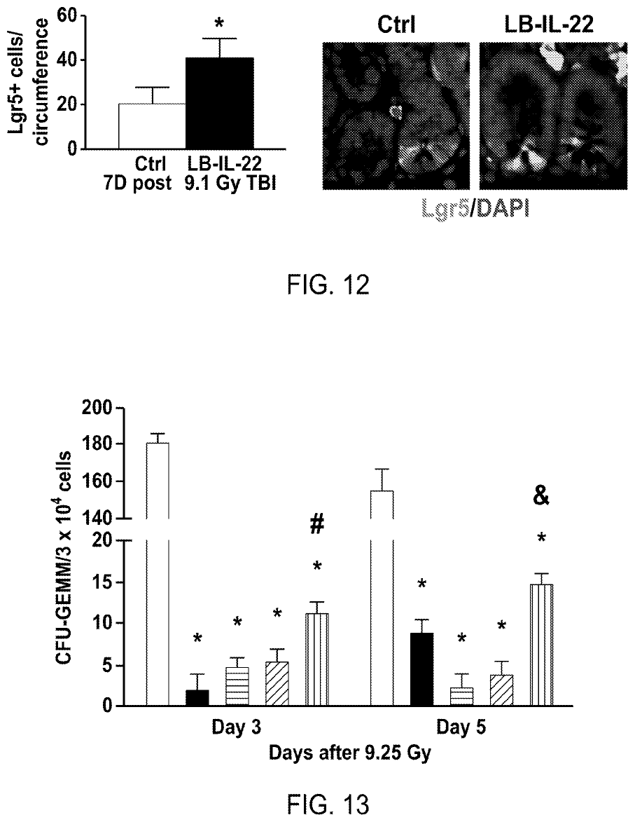

To better understand what the biological effect of L. reuteri-mediated delivery of IL-22 the intestinal crypt cells were analyzed. [Wei L, et al., Inhibition of CDK4/6 protects against radiation-induced intestinal injury in mice. J Clin Invest 126(11): 4076-4086, 2016; Wei L, et al., The GS-nitroxide JP4-039 improves intestinal barrier and stem cell recovery in irradiated mice. Scientific Rep 8: 2072, 2018.] Gavage of Lactobacillus-IL-22 at 24 h after irradiation was associated with increased numbers of lgr5+ crypt cells compared to the numbers detected in control irradiated mice (FIG. 12) There were also increased numbers of intestinal Paneth cells, intestinal goblet cells, and intestinal villus length at day 2, day 5, or day 7 after total body irradiation in the LR-IL-22 treated mice, which, collectively, demonstrates that recombinant IL-22 delivered by L. reuteri is biologically active. Control Lactobacillus had no protective effect.

### Example 10

### Gavage of L. Reuteri-IL22 Mitigates Intestinal as Well as Hematopoietic Irradiation Damage

C57BL/6NTac mice were irradiated to 9.25 Gy TBI. Twenty-four hours later the mice were injected I.M. with IL22 protein (0.1 mg/kg) or gavaged with either L. reuteri or L reuteri-IL22. On day 3 or 5 after irradiation, the mice were sacrificed and the bone marrow isolated and plated in methylcellulose media. Colonies of greater than 50 cells were counted 14 days later. All irradiated mice had significantly decreased number of colonies compared to non-irradiated mice (FIG. 13). This indicates a decrease in the number of hematopoietic progenitor cells following irradiation. Bone marrow from mice gavaged with L. reuteri-IL-22 had increased number of colonies compared to the bone marrow from 9.25 Gy mice. This demonstrates that gavage of L. reuteri-IL22 was able to partially mitigate irradiation-induced damage to the bone marrow by protecting the hematopoietic progenitor cells in the bone marrow.

### Example 11

### Optimization of Time of Delivery of Lactobacillus-IL-22

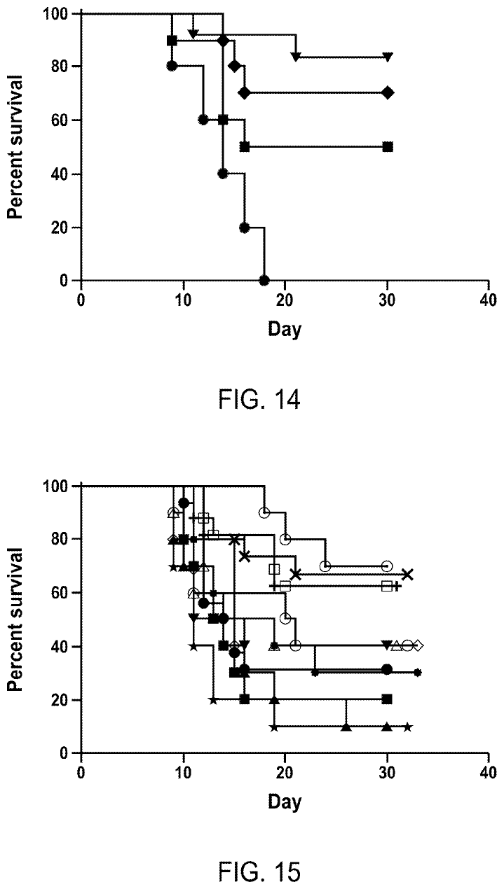

Given the data showing that recombinant L. reuteri can rescue mice from TBI induced death, the next experiment was to identify to what extent delivery of LR-IL-22 was effective at times later than 24 h after TBI. Administration of Lactobacillus-IL-22 was effective at all time points after TBI, but was most effective at mitigation if delivered at 24 hrs. (FIG. 14). While mitigators should be given as soon after TBI exposure as possible, these data indicate that mitigation can still be achieved by LR-IL-22 if delivered after 24 h.

### Example 12

### Standardization of the Number of Bacteria to be Administered to Obtain the Highest Mitigation

To better understand the dynamics of the irradiation mitigation by recombinant L. reuteri and E. coli, we conducted a dose-response experiment. C57BL/6NTac mice were irradiated to 9.25 Gy and 24 hrs later gavaged with Lactobacillus-IL-22 or E. coli-IL-22-GFP, or 48 hrs after irradiation with E. coli-IFN-β. The number of bacteria gavaged ranged from 10⁶ to 10⁹ cells (FIG. 15). Radiation mitigation was optimal for 10⁹ Lactobacillus-IL-22 and E. coli-IFN-β when mice were gavaged with 10⁹ cells. E. coli-IL-22 bacteria provided the best mitigation when 10⁷ cells were gavaged. There was a correlation of number of LR-IL-22 bacteria, which improved lgr5+ cell numbers and survival. In contrast, there was not a linear effect of number of E. coli-IL-22 with improved survival.

### Example 13

### Combined Treatment Using Microbial Therapeutic Probiotics and Small Molecule Radiation Mitigators

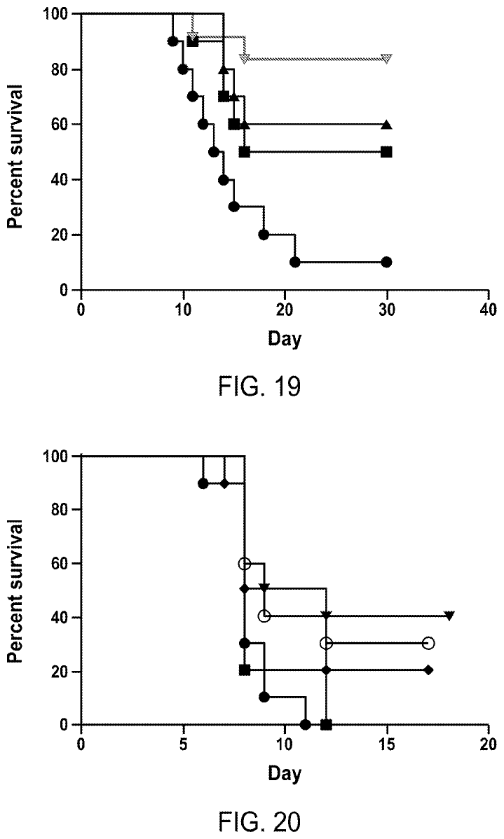

Enteric administration of Lactobacillus reuteri genetically modified to secrete IL-22 or Interferon-β, alone or in combination with parenteral administration of small molecule radiation mitigators ameliorated intestinal radiation damaged Paneth and lgr5+stem cells, restored barrier function, and regenerated stem cells. Enteric delivery of IL-22 by Lactobacillus reuteri-IL-22 circumvented the edema and impaired circulation in the intestine at 24 h after TBI, which limited effectiveness of parenterally administered IL-22. A combination of parenteral anti-apoptotic JP4-039 and enteric Lactobacillus reuteri-IL-22 demonstrates superior radiation mitigation compared to one category of mitigators alone (FIG. 19).

### Example 14

### Stabilization of Intestinal Lactobacillus Correlates with Successful Total Body Irradiation Mitigation

To determine whether successful radiation mitigation correlated with changes in the intestinal microbiome, groups of mice received 9.25 Gy total body irradiation (TBI) and subgroups treated with the small molecule radiation mitigator, JP4-039 (20 mg/kg in 50 microliters of 30% 2-hydroxypropyl-β-cyclodextrin I.M.), G-CSF (4 mg/kg I.P.), or both mitigators at 24 h after TBI. Fecal pellets were analyzed pre-irradiation, and at days 1, 3, 5, 7, 10, 14, 21, and 30 after irradiation. There was a significant improvement in survival of mice receiving either single mitigator or combination of two agents (30 days survival of 50% for JP4-039, 70% G-CSF, 80% for both drugs, compared to survival of irradiated controls of 20%, p =0.045, 0.0209 and 0.0060, respectively, vs radiation control at day 30, n=10). There was a clear correlation of relative abundance of the Lactobacillus taxon at day 14 with survival to day 30 in individuals in all treatment groups, as well as, in radiation controls, for all mice destined to survive to day 30 p=0.00244. There was also a correlation of survival with overabundance of Roseburia and Akkermansia.

### Example 15

### Genetically Engineered Probiotics Increase Survival Following 19.75 Gy Abdominal Irradiation

C57BL/6MTac were irradiated to 19.75 Gy to the abdomen using 6 MV photons. The remainder of the mouse was shielded from irradiation. Some mice were gavaged with 109 control Lactobacillus, Lactobacillus expressing murine IL-22, E. coli secreting murine IL-22, or E. coli secreting murine IFN-β-all gavages are in 100 ul PBS-3 separate gavages-at: 1) 24 hr before, 2) 1 hr after and 3) 24 hr after abdominal irradiation. The mice were followed for development of the GI-gastro-intestinal syndrome at which time they were sacrificed (weight-loss, failure to drink or eat, and listless or moribund). FIG. 20 shows that mice administered Lactobacillus-1L22, E. coli-IL-22, or E. coli-IFN-β had significantly increased survival.

### REFERENCES

Rwigema J -CM, Beck B, Wang W, Doemling A, Epperly M W, Shields D, Franicola D, Dixon T, Frantz M -C, Wipf P, Tyurina Y, Kagan V E, Wang H, and Greenberger J S: Two strategies for the development of mitochondrial-targeted small molecule radiation damage mitigators. Int J Radiat Oncol Biol Phys 80(3): 860-868, 2011.
Steinman J, Epperly M, Hou W, Willis J, Wang H, Fisher R, Liu B, Bahar I, McCaw T, Kagan V, Bayir H, Yu J, Wipf P, Li S, Huq M S, and Greenberger J S: Improved total-body irradiation survival by delivery of two radiation mitigators that target distinct cell death pathways. Radiat Res 189(1): 68-83, 2018. PMID: 29140165. DOI: 10.1667/RR14787.1.
Asano J, Sato T, Ichinose S, Kajita M, Onai N, Shimizu S, and Ohteki T: Intrinsic autophagy is required for the maintenance of intestinal stem cells and for irradiation-induced intestinal regeneration. Cell Rep 20(5): 1050-1060, 2017. PMID: 28768191. DOI: 10.1016/j.celrep.2017.07.019.

Wang X, Wei L, Cramer J M, Leibowitz B J, Judge C, Epperly M, Greenberger J, Wang F, Li L, Martin M G, Lagasse E, Zhang L, and Yu J: Pharmacologically blocking p53-dependent apoptosis protects intestinal stem cells and mice from radiation. Scientific Rep 5: 8566, 2015. PMID: 25858503. DOI: 10.1038/srep08566.

Wei L, Leibowitz B J, Wang X, Epperly M, Greenberger J, Zhang L, and Yu J: Inhibition of CDK4/6 protects against radiation-induced intestinal injury in mice. J Clin Invest 126(11): 4076-4086, 2016. PMID: 27701148. DOI: 10.1172/JCI88410.
Wei L, Leibowitz B J, Epperly M, Bi C, Li A, Steinman J, Wipf P, Zhang L, Greenberger J S, and Yu J: The GS-nitroxide JP4-039 improves intestinal barrier and stem cell recovery in irradiated mice. Scientific Rep 8: 2072, 2018. PMID: 29391546. DOI: 10.1038/s41598-018-20370-9.
Chen J, Huang C, Wang J, Zhou H, Lu Y, Lou L, Zhang J, Tian L, Wang X, Cao Z, and Zeng Y: Dysbiosis of intestinal microbiota and decrease in Paneth cell antimicrobial peptide level during acute necrotizing pancreatitis in rats. PLoS ONE 12(4): e0176583, 2017. PMID: 28441432. DOI: 10.1371/journal.pone.0176583.
Riba A, Olier M, Lacroix-Lamande S, Lencina C, Bacquie V, Harkat C, Gillet M, Baron M, Sommer C, Mallet V, Salvador-Cartier C, Laurent F, Theodorou V, and Menard S: Paneth cell defects induce microbiota dysbiosis in mice and promote visceral hypersensitivity. Gastroenterology 153: 1594-1606, 2017. PMID: 28865734. DOI: 10.1053/j.gastro.2017.08.044.
Wang J, Tian F, Wang P, Zheng H, Zhang Y, Tian H, Zhang L, Gao X, and Wang X: Gut microbiota as a modulator of Paneth cells during parenteral nutrition in mice. J Parenter Enteral Nutr 42(8): 1280-1287, 2018. PMID: 29701912. DOI: 10.1002/jpen.1162.

Guo X, Tang R, Yang S, Lu Y, Luo J, and Liu Z: Rutin and its combination with inulin attenuate gut dysbiosis, the inflammatory status, and endoplasmic reticulum stress in Paneth cells of obese mice induced by high-fat diet. Front Microbiol 9: 2651, 2018. PMID: 30455677. DOI: 10.3398/fmicb.2018.02651.

Gassier N: Paneth cells in intestinal physiology and pathophysiology. World J Gastrointest Pathophysiol 8(4): 150-160, 2017. PMID: 29184701. DOI: 10.4291/wjgp.v8.i4.150.

Dayton T L, and Clevers H: Beyond growth signaling: Paneth cells metabolically support ISCs. Cell Res 17: 851-852, 2017. PMID: 28429766. DOI: 10.1038/cr.2017.59.

Langlands A J, Almet A A, Appleton P L, Newton I P, Osborne J M, and Nathke I S: Paneth cell-rich regions separated by a cluster of Lgr5+ cells initiate crypt fission in the intestinal stem cell niche. PLoS Biol 14(6): e1002491, 2016. PMID: 27348469. DOI: 0.1371/journal.pbio.1002491.

Bel S, Pendse M, Wang Y, Li Y, Ruhn K A, Hassell B, Leal T, Winter S E, Xavier R J, and Hooper L V: Paneth cells secrete lysosome via secretory autophagy during bacterial infection of the intestine. Science 357(6355): 1047-1052, 2017.
Rogala A R, Schoenborn A A, Fee B E, Cantillana V A, Joyce M J, Gharaibeh R Z, Roy S, Fodor A A, Sartor R B, Taylor G A, and Gulati A S: Environmental factors regulate Paneth cell phenotype and host susceptibility to intestinal inflammation in Irgml-deficient mice. Dis Model Mech 11(2): xx, 2018. PMID: 29361512. DOI: 0.1242/dmm 031070.
Yu S, Tong K, Zhao Y, Balasubramanian I, Yap G S, Ferraris R P, Bonder E M, Verzi M P, and Gao N: Paneth cells multipotency induced by notch activation following injury. Cell Stem Cell 23(1): 46-59, 2018. PMID: 29887318. DOI: 10.1016/j.stem.2018.05.002.
Zha J -M, Li H -S, Lin Q, Kuo W -T, Jiang Z -H, Tsai P -Y, Ding N, Wu J, Xu S -F, Wang Y -T, Pan J, Zhou X -M, Chen K, Tao M, Odenwald M A, Tamura A, Tsukita S, Turner J R, and He W -Q: Interleukin 22 expands transit-amplifying cells while depleting Lgr5+ stem cells via inhibition of Wnt and notch signaling. Cell Mol Gastroeneterol Hepatol 7(2): 255-274, 2018. PMID: 30686779. DOI: 10.1016/j.jcmgh.2018.09.006.
Thomas L S, Targan S R, Tsuda M, Yu Q T, Salumbides B C, Haritunians T, Mengesha E, McGovern D P B, and Michelsen K S: The TNF family member TL1A induces IL-22 secretion in committed human TH17 cells via IL-9 induction. Journal of Leukocyte Biology 101: 727, 2017. PMID: 27733581. DOI: 10.1189/jlb.3A0316-129R.
Zhao K, Ruan S, Tian Y, Zhao D, Chen C, Pan B, Yan Z, Yin L, Zhu S, and Xu K: IL-22 promoted CD3+T cell infiltration by IL-22R induced STAT3 phosphorylation in murine acute graft versus host disease target organs after allogeneic bone marrow transplantation. International Immunopharmacology 39: 383-388, 2016. PMID: 27551984. DOI: 10.1016/j.intimp.2016/08.012.
Pan B, Xia F, Wu Y, Zhang F, Lu Z, Fu R, Shang L, Li L, Sun Z, Zeng L, and Xu K: Recipient-derived IL-22 alleviates murine acute graft-versus-host disease in association with reduced activation of antigen presenting cells. Cytokine 111: 33-40, 2018. PMID: 30114627. DOI: 10.1016/j.cyto.2018.08.010.
Aden K, Tran F, Ito G, Sheibani-Tezerji R, Lipinski S, Kulper J W, Tschurtschenthaler M, Savelijeva S, Bhattacharyya J, Hasier R, Bartsch K, Luzius A, Jentzsch M, Falk-Paulsen M, Stengl S T, Wetz L, Schwarzer R, Rabe B, Barchet W, Krautwald S, Hartmann G, Pasparaki M, Blumberg R S, Schreiber S, Kaser A, and Rosenstiel P: ATG16L1 orchestrates Interleukin-22 signaling in the intestinal epithelium via cGAS-STING. J Exp Med 215(11): 2868-2886, 2018. PMID: 30254094. DOI: 10.1084/jem.20171029.
Aden K, Tran F, Ito G, Sheibani-Tezerji R, Lipinski S, Kulper J W, Tschurtschenthaler M, Savelijeva S, Bhattacharyya J, Hasier R, Bartsch K, Luzius A, Jentzsch M, Falk-Paulsen M, Stengl S T, Wetz L, Schwarzer R, Rabe B, Barchet W, Krautwald S, Hartmann G, Pasparaki M, Blumberg R S, Schreiber S, Kaser A, and Rosenstiel P: ATG16L1 orchestrates Interleukin-22 signaling in the intestinal epithelium via cGAS-STING. J Exp Med 215(11): 2868-2886, 2018 PMID: 30700914. DOI: 10.10381s41586-019-0899-7.
Bootz F, Ziffels B, and Neri D: Antibody-based targeted delivery of interleukin-22 promotes rapid clinical recovery in mice with DSS-induced colitis. Inflamm Bowel Dis 22(9): 2098, 2016. PMID: 27482975. DOI: 10.107/MIB.0000000000000851.
Hammer A M, Morris N L, Cannon A R, Khan O M, Gagnon R C, Movichan N V, van Langeveid I, Li X, Gao B, and Choudhry M A: Interleukin-22 prevents microbial dysbiosis and promotes intestinal barrier regeneration following acute injury. Shock 48(6): 657-665, 2017. PMID: 28498296. DOI: 10.1097/SHK.0000000000000900.
Eyerich K, Dimartino V, and Cavani A: IL-17 and IL-22 in immunity: driving protection and pathology. Eur J Immunol 47: 607-614, 2017. PMID: 28295238. DOI: 10.1002/eji.201646723.
Hu M, Lin H, Yang L, Cheng Y, and Zhang H: Interleukin-22 restored mitochondrial damage and impaired glucose-stimulated insulin secretion through down-regulation of uncoupling protein-2 in INS-1 cells. J Biochem 161(5): 433-439, 2017. PMID: 28069865. DOI: 10.1093/jb/mvw084.
Khawar M B, Azam F, Sheikh N, and Mujeeb K A: How does interleukin-22 mediate liver regeneration and prevent injury and fibrosis? J Immunol Res 2016: 2148129, 2016. PMID: 28050571. DOI: 10.1155/2016/2148129.
Valeri M, and Raffatellu M: Cytokines IL-17 and IL-22 in the host response to infection. Pathog Dis 74(9): 1, 2016. PMID: 27915228. DOI: 10.1093/femspd/ftw111.
Huang Z, Epperly M, Watkins S C, Greenberger J S, Kagan V E, Bayir H: Necrostatin-1 rescues mice from lethal irradiation. Biochimica at Biophysica Acta 1862: 850-856, 2016. PMID: 26802452. DOI: 10.1016/j.bbadis.2016.01.014.
Vanden Berghe T, Linkermann A, Jouan-Lanhouet S, Walczak H, and Vandenabeele P: Regulated necrosis: the expanding network of non-apoptotic cell death pathways. Nature Reviews Molecular Cell Biology 15: 135-147, 2014. PMID: 24452471. DOI: 10.1038/nrm3737.
Epperly M W, Wipf P, Fisher R, Franicola D, Beumer J, Li S, Brand R, Falo L, Greenberger J S: Evaluations of different formulations and routes for the delivery of the ionizing radiation mitigation GS-nitroxide (JP4-039). In Vivo 32: 1009-1023, 2018.
Goudarzi M, Mak T D, Jacobs J P, Moon B -H, Strawn S J, Braun J, Brenner D J, Fornace, Jr. A J, and Li H -H: An integrated multi-omic approach to assess radiation injury on the host-microbiome axis. Radiat Res 186: 219-234, 2016. PMID: 27512828. DOI: 10.1667/RR14306.1.
Packey C D, and Ciorba M A: Microbial influences on the small intestinal response to radiation injury. Curr Opin Gastroenterol 26(2): 88-94, 2010. PMID: 20040865. DOI: 10.1097.MOG.0b013e3283361927.
Kim Y S, Kim J, and Park S -J: High-throughput 16S rRNA gene sequencing reveals alterations of mouse intestinal microbiota after radiotherapy. Anaerobe 33: 1-7, 2015. PMID: 25600706. DOI: 10.1016/j.anaerobe.2015.01.004.
Meisel M, Hinterleitner R, Pacis A, Chen L, Earley Z M, Mayassi T, Pierre J F, Ernest J D, Galipeau H J, Thuille N, Bouziat R, Buscarlet M, Ringus D L, Wang Y, Li Y, Dinh V, Kim S M, McDonald B D, Zurenski M A, Musch M W, Furtado G C, Lira S A, Baier G, Change E B, Eren A M, Weber C R, Busque L, Godley L A, Verdu E F, Barreiro L B, and Jabri B: Microbial signals drive pre-leukaemic myeloproliferation in a Tet2-deficient host. Nature 447: 20-30, 2018. PMID: 29769727. DOI: 10.1038/s41586-018-0125-z.
Hendrikx T, Duan Y, Wang Y, Oh J -H, Alexander L M, Huang W, Starkel P, Ho S B, Gao B, Fiehn O, Emond P, Sokol H, van Pijkeren J P, and Schnabl B: Bacteria engineered to produce IL-22 in intestine induce expression of REG3G to reduce ethanol-induced liver disease in mice. Gut Epub ahead of print Nov. 17, 2018, doi: 10.1136/gutjnl-2018-317232.
Tyurina Y Y, St. Croix C, Watkins S, Epperly M, Anthonymuthu T, Kisin E, Vlassova I I, Krysko O, Krysko D, Kapralov A, Dar H, Tyurin V A, Popova E N, Bolevich S B, Timashev P S, Kellum J A, Wenzel S E, Mallampalli R, Greenberger J S, Bayir H, Shvedova A A, and Kagan V E: Redox (phospho)lipidomics of signaling in inflammation and programmed cell death. J Leukocyte Biol 106: 57-81, 2019. PMID: 31071242. DOI: 10.1002/JLB.3MIR0119-004RR.
van Pijkeren J -P, Britton R A: High efficiency recombineering in lactic acid bacteria.Nucleic Acids Res 40: e76, 2012. PMID: 22328729. DOI: 10.1093/nar/gks147.
Alexander L M, Oh J -H, Stapleton D S, Schueler K L, Keller M P, Attie A D, van Pijkeren J -P: Exploiting prophage-mediated lysis for biotherapeutic release by Lactobacillus reuteri. Appl Environ Microbiol 85: e02335-18, 2019. PMID: 30683744. DOI: 10.1128/AEM.02335-18.
Fu X, Xu J -G: Development of a chromosome-plasmid balanced lethal system for Lactobacillus acidophilus with thyA gene as selective marker. Microbiol Immunol 44: 551-556, 2000. PMID: 10981827. DOI: 10.1111/j.1348-0421.2000.tb02533.x.
Hendrikx T, Duan Y, Wang Y, Oh J -H, Alexander L M, Huang W, Starkel P, Ho S B, Gao B, Fiehn O, Emond P, Sokol H, van Pijkeren J -P, Schnabl B: Bacteria engineered to produce IL-22 in intestine induce expression of REG3G to reduce ethanol-induced liver disease in mice. Gut gutjnl pg. 317232, 2018. PMID: 30448775. DOI: 10.1136/gutjnl-2018-317232.
Leenhouts K, Buist G, Bolhuis A, ten Berge A, Kid J, Mierau I, Dabrowska M, Venema G, Kok J: A general system for generating unlabeled gene replacements in bacterial chromosomes. Mol Gen Genet MGG 253: 217-224, 1996.

### Greenberger J S: “Radioprotection”. In Vivo 23: 323-336, 2009.

Greenberger J S, Kagan V E, Pearce L, Boriseniao G, Tyurina Y, and Epperly M W: Modulation of redox signal transduction pathways in the treatment of cancer. Antioxidants Redox Signaling 3(3): 347-359, 2001. PMID: 11491649. DOI: 10.1089/15230860152409004.
Greenberger J S, Epperly M W: Radioprotective antioxidant gene therapy: potential mechanisms of action. Gene Therapy and Molecular Biology (GTMB) 8: 31-44, 2004.
Stone H B, Coleman C N, Moulder J E, Ang K K, Anscher M S, Barcellos-Hoff M H, Dynan W S, Fike J R, Grdina D J, Greenberger J S, Hauer-Jensen M, Hill R P, Kolesnick R N, MacVittie T J, Marks C, McBride W H, Metting N, Pellmar T, Purucker M, Robbins M E C, Schiestl R H, Seed T M, Tomaszewski J, Travis E L, Wallner P E, Wolpert M, Zaharevitz D: Models for evaluating agents intended for the prophylaxis, mitigation, and treatment of radiation injuries. Report of an NCI Workshop, Dec. 3-4, 2003. Radiat Res 162: 711-718, 2004. PMID: 15548121. DOI: 10.1667/rr3276.
Tyurina Y Y, Shrivastava I, Tyurin V A, Mao G, Dar H, Watkins S, Epperly M, Bahar I, Shvedova A A, Pitt B, Mallampalli R, Sadovsky Y, Gabrilovich D, Greenberger J S, Bayir H, Kagan V E. “Only a life lived for others is worth living:” Redox signaling by oxygenated phospholipids in cell fate decisions. Antioxidant and Redox Signaling,
Song X, Xie Y, Kang R, Hou W, Sun X, Epperly M W, Greenberger J S, Daolin T. FANCD2 protects against bone marrow injury from ferroptosis. Biochem and Biophys Com 480(3): 443-449, 2016
Krainz T, Du L, Lewis T, Lamade S M, Calderon M, Watkins S C, Epperly M W, Greenberger J S, Bayir H, Wipf P, Clark R S B. Synthesis and evaluation of a mitochondria-targeting Poly(ADP-ribose) Polymerase-1 inhibitor. ACS, Chem Biol, 13(10): 2868-2878, 2018
Coll R C, Robertson A A B, Chae J J, Higgins S C, Munoz-Planillo R, Inserra M C, Vetter I, Dungan L S, Monks B G, Stutz A, Croker D E, Butler M S, Haneklaus M, Sutton C E, Nunez G, Latz E, Kastner D L, Mills K H G, Masters S L, Schroder K, Cooper M A, O'Neill L A J. A small-molecule inhibitor of the NLRP3 inflammasome for the treatment of inflammatory diseases. Nature Medicine 21(3): 248-255, 2015
Gloor G B, Macklaim J M, Pawlowsky-Glahn V, Egozcue J J. Microbiome datasets are compositional: And this is not optional. Front Microbiol 8: 2224, 2017
Tarabichi Y, Li K, Hu S, Nguyen C, Wang X, Elashoff D, Methe B A. The administration of intranasal live attenuated influenza vaccine induces changes in the nasal microbiota and nasal epithelium gene expression profiles. Microbiome 1(3): 1-16, 2015
Stoyanovsky D A, Vlasova I I, Belikova N A, Kapralov A, Tyurin V, Greenberger J S, Kagan V E. Targeting and activation of NO donors in mitochondria. Peroxidase metabolism of (2-Hydroxyamino-Vinyl)-Triphenyl-Phosphonium by cytochrome c releases NO and protects cells from apoptosis. FEBJ Letters 583: 2000-2005, 2009
Christner S, Guo J, Praise R A, Ringeval M, Hoye A T, Wipf P, Epperly M, Greenberger J S, Beumer J H, Eiseman J L. Liquid chromatography-tandem mass spectrometric assay for the quantitation of the novel radiation protective agent and radiation mitigator JP4- 039 in murine plasma. J Pharmaceut and Biomed Anal S0731-7084(17): 32605-5, 2017
Roth D, Chiang A J, Hu W, Gugiu G B, Morra C N, Versalovic J, Kalkum M. Two-carbon folate cycle of commensal Lactobacillus reuteri 6475 gives rise to immunomodulatory ethionine, a source for histone ethylation. FASEB J, 33: 3536-3548, 2019

1. A method of treating an irradiation-induced intestinal damage in a subject comprising administering to the subject a therapeutically effective amount of a gastrointestinal tract (GI) bacterium, wherein the bacterium comprises a vector comprising a polynucleotide that encodes IL-22 and/or IFN-β, or a functional fragment thereof.

2. The method of claim 1, wherein the GI bacterium is a specie of Escherichia genus or Lactobacillus genus.

3. The method of claim 2, wherein the GI bacterium is Lactobacillus reuteri.

4. The method of claim 2, wherein the GI bacterium is Escherichia coli.

5. The method of claim 1, wherein the GI bacterium resides in an area of a colon or a small intestine following administration.

6. The method of claim 1, wherein the GI bacterium secretes IL-22 and/or IFN-β, or a functional fragment thereof.

7. The method of claim 1, wherein the vector comprises SEQ ID NO: 4.

8. The method of claim 1, wherein the vector comprises SEQ ID NO: 2.

9. The method of claim 1, wherein the subject has a cancer and has received or is intended to receive an irradiation therapy.

10. The method of claim 9, wherein the cancer is an ovarian cancer.

11. The method of claim 9, wherein the irradiation therapy comprises total body irradiation or abdominal irradiation.

12. The method of claim 1, wherein the GI bacterium is administered to the subject via an oral administration.

13. The method of claim 1, further comprising administering to the subject a therapeutically effective amount of an irradiation mitigator.

14. The method of claim 13, wherein the irradiation mitigator is JP4-039, Necrostatin, Baicalein, XJB-Veliparib, triphenyl-phosphonium-Veliparib, MCC950, or G-CSF.

15. The method of claim 1, wherein the treatment results in a reduction of an intestinal damage caused by irradiation.

16. The method of claim 1, wherein the administration is a dosage from about 1×106 to about 1×109 colony forming units (CFU) of the bacterium.

17. A gastrointestinal tract (GI) bacterium comprising a vector, wherein the vector comprises a polynucleotide that encodes IL-22 or a functional fragment thereof, and wherein the bacterium secretes IL-22 or a functional fragment thereof, and wherein the GI bacterium is a species of Escherichia genus.

18. A gastrointestinal tract (GI) bacterium comprising a vector, wherein the vector comprises a polynucleotide that encodes IFN-β or a functional fragment thereof, and wherein the bacterium secretes IFN-β or a functional fragment thereof.

19. The GI bacterium of claim 18, wherein the GI bacterium is a species of Escherichia genus or Lactobacillus genus.

20. The GI bacterium of claim 19, wherein the GI bacterium is Lactobacillus reuteri.

21. The GI bacterium of claim 19, wherein the GI bacterium is Escherichia coli.
